(19) United States
(12) Patent Application Publication
(10) Pub. No.: US 2026/0369402 A1
(43) Pub. Date: Nov. 19, 2026

(54) IDENTIFYING A SUBJECT SUFFERING FROM ALZHEIMER'S DEMENTIA OR BEING AT RISK OF DEVELOPING ALZHEIMER'S DEMENTIA

(71) Applicant: Immungenetics AG, Hamburg (DE)

(72) Inventor: Markus KROHN, Heidelberg (DE)

(21) Appl. No.: 19/121,972

(22) PCT Filed: Oct. 17, 2023
(86) PCT No.: PCT/EP2023/078829

§ 371 Date: Apr. 17, 2025

(30) Foreign Application Priority Data
Oct. 18, 2022 (EP) 22202129.7

(51) Int. Cl. G16H 50/20; G16B 40/10; G16B 40/20; G16H 50/30; G16H 50/70

(52) U.S. Cl. CPC G16H 50/20; G16B 40/10; G16B 40/20; G16H 50/30; G16H 50/70

(57) ABSTRACT

In a first aspect the invention relates to a method for identifying a subject suffering from Alzheimer's dementia (AD) or being at risk of developing Alzheimer's dementia, the method comprising: (a) determining in a first body fluid sample of the subject the amount of at least a compound having a mass in gram/mol of 110.0367; (b) determining in a second body fluid sample of the subject the amount of the same compound as in (a); (c) determining the change of the amount of the compound determined according to (a) compared to the respective amount of the compound determined according to (b); (d) analyzing the change as determined in (c) using a computer-implemented prediction algorithm that is capable of predicting AD in a subject based on the compound, whereby a subject suffering from AD or being at risk of developing Alzheimer's dementia is identified if AD is predicted; wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

In a first aspect, the invention relates to a method for identifying a subject suffering from Alzheimer's dementia (AD) or being at risk of developing Alzheimer's dementia, the method comprising: (a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1); (b) analyzing the determined amount for the at least one compound using a computer-implemented prediction algorithm that is capable of predicting AD in a subject based on the at least one compound, whereby a subject suffering from AD or being at risk of developing Alzheimer's dementia is identified if AD is predicted. In a second aspect the invention relates to a method for identifying a subject suffering from Alzheimer's dementia (AD) or being at risk of developing Alzheimer's dementia. A third aspect of the invention and a fourth aspect of the invention both relate to methods for identifying a subject suffering from Alzheimer's dementia (AD) or being at risk of developing Alzheimer's dementia. In a fifth aspect, the invention relates to a second diagnostic use, and a sixth aspect of the invention is directed to a device adapted for identifying a subject suffering from Alzheimer's dementia (AD) or being at risk of developing Alzheimer's dementia, whereas a seventh aspect of the invention is related to a kit for identifying a subject suffering from Alzheimer's dementia (AD) or being at risk of developing Alzheimer's dementia.

### STATE OF THE ART

The accumulation of proteins or protein fragments (peptides) in the brain is a significant feature of age-related neurodegenerative diseases. In Alzheimer's disease (AD) and cerebral β-amyloid angiopathy (CAA), the aggregation of β-amyloid peptides (Aβ) is considered to trigger the disease. Regarding treatment of AD, several active agents are known, such as, for example, Thiethylperazine [1]. Regarding diagnosis, higher amounts of Aβ peptides of 40 and 42 amino acids in the brain are partly reflected by an increase of these peptides in the peripheral blood stream and are utilized as diagnostic indicator for Alzheimer's disease. However, quantitation of Aβ in blood samples is unreliable even using extremely sensitive methods like liquid chromatography coupled with mass spectrometry (LC-MS/MS) and requires Aβ immunoprecipitation as an additional and expensive step of sample preparation. Such assays have become publicly available recently and achieve an accuracy of about 77 percent in predicting an AD indicative brain amyloid status in the age group of 65-75 years old patients with a declining accuracy with lowering age [2]. Another study of E. Trushina et al. using UPLCToF-MS-based metabolomics analyzed and compared metabolite profiles in plasma and cerebrospinal fluid samples from cognitive normal probands, probands having mild cognitive impairment and AD patients [3]. However, whilst early stage diagnosis is mentioned as kind of a catchword, it is also pointed out that the study at the current stage is not able to do more than to differentiate between healthy and ill probands. In addition there are further differences such as which kind of bodily fluid is investigated since there are different metabolic markers detected in plasma and in cerebrospinal fluid.

However, since it is hypothesized that the AD pathology is starting up to 20 years before the onset of clinical symptoms, diagnostic methods for detection of signs of AD pathology in the younger population are needed.

The technical problem underlying thus was the provision of means and methods for complying with the aforementioned needs, especially to provide a method for diagnosing or pre-diagnosing Alzheimer's dementia in a subject.

1^{st} Aspect—Method for Identifying a Subject Suffering from Alzheimer's Dementia (AD) or being at Risk of Developing Alzheimer's Dementia

Accordingly, the present invention relates in a first aspect to a method for identifying a subject suffering from Alzheimer's dementia (AD) or being at risk of developing Alzheimer's dementia, the method comprising:
(a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
(b) analyzing the determined amount for the at least one compound using a computer-implemented prediction algorithm that is capable of predicting AD in a subject based on the at least one compound, whereby a subject suffering from AD or being at risk of developing Alzheimer's dementia is identified if AD is predicted.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The term “identifying a subject suffering from Alzheimer's dementia (AD)” is synonymous with “diagnosing Alzheimer's dementia (AD) in a subject”, wherein “identifying” as well as “diagnosing” as used herein both refer to assessing whether a Ka disease or not. Accordingly, the presence or the absence of a disease in the subject can be diagnosed. Preferably, the term refers to ruling in or ruling out said disease. As will be understood by those skilled in the art, such an assessment, although preferred to be, may usually not be correct for 100% of the investigated subjects. The term, however, requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, and so on. Details can, for example, be found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2 or lower, 0.1 or lower, or 0.05 or lower.

The terms “identifying” as well as “diagnosing” include individual diagnosis of a disease or its symptoms as well as continuous monitoring of a patient. Monitoring, i.e. diagnosing the presence or absence of a disease or the symptoms accompanying it at various time points, includes monitoring of patients known to suffer from the disease as well as monitoring of subjects known to be at risk of developing the disease, e.g. subjects with, for example, a genetic predisposition for the disease or subjects who have been determined as being at risk based on the above-identified method. Furthermore, monitoring can also be used to determine whether a patient is treated successfully or whether at least symptoms of the disease can be ameliorated over time by a certain therapy. Moreover, monitoring may be used for active patient management including deciding on hospitalization, intensive care measures and/or additional qualitative monitoring as well as quantitative monitoring measures.

The term “identifying a subject being at risk of developing Alzheimer's dementia” refers to assessing the probability according to which a subject will suffer from Alzheimer's dementia within a certain time window, i.e. the predictive window. The term, preferably, relates to predicting whether or not there is an increased risk for Alzheimer's dementia compared to the average risk for developing Alzheimer's dementia in a population of subjects rather than giving a precise probability for the said risk.

Alzheimer's dementia (AD) (Morbus Alzheimer, Alzheimer's disease) is a neurodegenerative disease that is characterized by increasing dementia. Methods known so far for determining whether a subject is suffering from AD or not, are current diagnostic standards, preferably based on amyloid positron emission tomography and/or cerebrospinal fluid diagnostics and/or through cognitive testing. Biomarkers relevant for AD are biomarkers of neurodegeneration, which are known to the skilled person, like for example, Abeta-40, Abeta-42, or Abeta42/40 ratio, phosphorylated variants of the Tau-protein (e.g. p-Tau181, p-Tau217 etc.), Glial fibrillary acidic protein (GFAP), Neurofilament light chain (NfL) etc. in bodily fluids (blood, CSF). Methods for determining whether a subject is suffering from AD are related to determination of the cognitive abilities by one or more tests, which are known to the skilled person, like for example, ADAS-Cog, FCSRT, ADCS-iADL, QOL-AD, CDR, MMSE etc. in a subject.

The term “subject”, as used herein, relates to a multicellular animal, preferably to a vertebrate, more preferably to a mammal, more preferably a human.

A “body fluid sample” is a sample from the group consisting of blood sample, urine sample, liquor sample (CSF) and mixtures of two or more thereof, preferably the body fluid sample is a blood sample.

The method for identifying a subject suffering from Alzheimer's dementia (AD) or being at risk of developing Alzheimer's dementia is an in vitro method.

The term “determining the amount” means a determination based on mass spectrometry (MS). Mass spectrometry as used herein encompasses all techniques which allow for the determination of the molecular weight (i.e. the mass) or a mass variable corresponding to a compound, i.e. a metabolic analyte. More preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or GC-MS, i.e. to mass spectrometry being operatively linked to a prior chromatographic separation step. More preferably, mass spectrometry as used herein encompasses LC-MS/MS (Liquid-Chromatography-Mass spectrometry/Mass spectrometry). Liquid chromatography as used herein refers to all techniques which allow for separation of compounds (e.g. of the at least two metabolic analytes to be determined in accordance with the present invention) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. A specific compound and its specific mass respectively as well as its amount are determined based on the related peak in a mass spectrum. Such a peak contains characteristic information of the compound, i.e. the m/z information, as well as an intensity value being related to the abundance of the said compound (i.e. its amount) in the sample.

In some preferred embodiments of the method for identifying a subject suffering from Alzheimer's dementia (AD) or being at risk of developing Alzheimer's dementia, the least one compound having a mass in gram/mol is selected from the group consisting of 110.0367, 232.004, 224.084, 254.1153, 189.9936, and 189.0096 (Group 2).

In some preferred embodiments of the method for identifying a subject suffering from Alzheimer's dementia (AD) or being at risk of developing Alzheimer's dementia, at least a compound having a mass in gram/mol of 110.0367 is determined in (a) and the determined amount thereof is analysed in (b).

In some preferred embodiments of the method for identifying a subject suffering from Alzheimer's dementia (AD) or being at risk of developing Alzheimer's dementia, the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in step (a) and analysed in (b), wherein more preferably one of the at least two compounds, preferably one of the at least three compounds, more preferably one of the at least four compounds, more preferably one of the at least five compounds is the compound having a mass in gram/mol of 110.0367.

In some preferred embodiments of the method for identifying a subject suffering from Alzheimer's dementia (AD) or being at risk of developing Alzheimer's dementia, the amount of at least two compounds, preferably at least six compounds, more preferably at least seven compounds, having a mass in gram/mol selected from the group consisting of Group 1 or Group 2 is determined in a first body fluid sample of the subject in step (a) and analysed in (b), wherein more preferably one of the at least two compounds, more preferably one of the at least six compounds or one of the at least seven compounds, is the compound having a mass in gram/mol of 110.0367.

In some preferred embodiments of the method for identifying a subject suffering from Alzheimer's dementia (AD) or being at risk of developing Alzheimer's dementia, the amount of at least one further compound having a mass in gram/mol selected from the group consisting of 208.0737, 315.2409, 175.0633, 702.5682, 161.0477, and 97.9769 (Group 3), preferably from the group consisting of 208.0737, 315.2409, 175.0633, 702.5682 (Group 4), is additionally determined in a first body fluid sample of the subject in steps (a) and additionally analysed in (b).

In the first aspect, the invention also relates to a method for identifying a subject suffering from Alzheimer's dementia (AD) or being at risk of developing Alzheimer's dementia, the method comprising:
(a) determining in a first body fluid sample of the subject the amounts of at least two compounds having a mass in gram/mol selected from the group consisting of Group 3, preferably of Group 4;
(b) analyzing the determined amounts for the at least two compounds using a computer-implemented prediction algorithm that is capable of predicting AD in a subject based on the at least two compounds, whereby a subject suffering from AD or being at risk of developing Alzheimer's dementia is identified if AD is predicted.

In some preferred embodiments of the method for identifying a subject suffering from Alzheimer's dementia (AD) or being at risk of developing Alzheimer's dementia, the first body fluid sample derives from a subject after the subject had been administered thiethylperazine or a pharmaceutically acceptable salt thereof, wherein the sample was preferably taken within 0.5 hours to 8 hours after TEP administration.

In some preferred embodiments of the method for identifying a subject suffering from Alzheimer's dementia (AD) or being at risk of developing Alzheimer's dementia, said computer-implemented prediction algorithm uses a supervised machine-learning algorithm that has been trained by a training dataset comprising quantitative or qualitative data for
the at least one, preferably at least two, more preferably at least three, more preferably at least four, more preferably at least five, compound(s) from Group 1, or Group 2, optionally in combination with the at least one further compound from Group 3; or optionally in combination with the at least one further compound from Group 4, or
the at least two, more preferably at least six, more preferably at least seven, compound(s) from Group 1, or Group 2 optionally in combination with the at least one further compound from Group 3, or optionally in combination with the at least one further compound from Group 4; or
the at least two compounds from Group 3, preferably from Group 4;
in body fluid samples from subjects known to suffer from AD and subjects known not to suffer from AD.

As indicated above, whether a subject is suffering from AD or not, is determined according to current diagnostic standards, preferably based on amyloid positron emission tomography and/or cerebrospinal fluid diagnostics and/or through cognitive testing.

The term “computer-implemented prediction algorithm” as used herein refers to a prediction algorithm that is carried out automatically by a data processing device such as a computer. The said data processing device, preferably, comprises tangibly embedded rules for automatically carrying out the prediction algorithm based on the measurement results obtained in the method of the invention. Said measurement results may be, preferably, stored in a database which can be permanently or temporarily linked to the data processor. Moreover, the linkage may be, preferably, a direct linkage by wire or a wireless linkage. The prediction algorithm as referred to herein is an algorithm which determines a parameter that is associated with the likelihood for a subject of suffering from AD. The prediction algorithm as referred to herein shall generate the aforementioned parameter and may process it further in order to establish a diagnostic result in accordance with the method of the invention. Preferably, the (computer-implemented prediction) parameter can be determined as set forth elsewhere herein. In some preferred embodiments of the method for identifying a subject suffering from Alzheimer's dementia (AD) or being at risk of developing Alzheimer's dementia, said supervised machine-learning algorithm establishes a classifying algorithm selected from the group consisting of Logistic Regression, Stochastic Gradient Descent Classifier (SGDClassifier), Decision Tree classifier, Random Tree classifier, Extra Tree classifier, AdaBoost Classifier, Multi-layer Perceptron classifier, Gradient Boosting Classifier, Gaussian Naïve Bayes, K Nearest Neighbor classifier, C-Support Vector Classification, Quadratic Discriminant Analysis, and Linear Regression. In some preferred embodiments of the method for identifying a subject suffering from Alzheimer's dementia (AD) or being at risk of developing Alzheimer's dementia, said supervised machine-learning algorithm establishes and/or applies a logistic regression model said logistic regression model being based on equitation (I)

y=I+C₁*F₁+C₂*F₂+ . . . +Cₙ*Fₙ, (I)
wherein I is the Intercept and Cₙ the coefficient of Mass Fₙ.

The computer-implemented prediction parameter here is y. Positive y-values indicate a likelihood for a subject of suffering or not suffering from AD, i.e. are a prediction of the respective condition. It will be understood that this is the case when training was done based on the definition that an AD diagnosis is the positive outcome, hence greater absolute values mean greater certainty of the prediction, i.e. a greater positive y value indicates a higher likelihood for a subject of suffering from AD while a negative value for y with a higher absolute value indicates a higher likelihood for a subject of not having AD. On the other hand, in case that training was done based on the definition that an AD diagnosis is the negative outcome, then a greater negative y-value indicates a higher likelihood for a subject of suffering from AD, while a positive value for y with a higher (absolute) value indicates a higher likelihood for a subject of not having AD.

Each coefficient has preferably a value in a range as indicated in Table 2 in the experimental part, wherein each range excludes zero-see for each specific mass combination the related line called “Ranges” in Table 2. For example, for a combination of the masses, each in gram/mol of 110.0367, 254.1153, 189.0096, and 224.084, the Intercept I has a value in the range of from −50 to +50, excluding zero, and the coefficient C1 If the first feature (i.e. of mass=110.0367/gram/mol is in the range of from −150 to +150, excluding zero etc. Furthermore, it is even more preferred, if the coefficients of the respective masses have a ratio to each other in the ranges indicated below in Table A, more preferred as indicated below in Table B, more preferred as indicated below in Table C, wherein these ratios more preferably apply for combinations of masses from Group 2, optionally in combinations with masses of Group 4:

TABLE A

| | 702.5682 | 189.0096 | 189.9936 | 110.0367 | 232.004 | 175.0633 | 254.1153 | 208.0737 | 315.2409 | 224.084 | |
|---|---|---|---|---|---|---|---|---|---|---|---|
| 702.5682 | // | 1218 | 468 | 257 | 341 | 15 | 307 | 11 | 242 | 538 | Upper |
| 189.0096 | −585 | // | 5372 | 24 | 4648 | 5 | 54 | 39446 | 507 | 612 | half = |
| 189.9936 | −302 | −322 | // | 930 | 3527 | 2 | 85 | 39637 | 266 | 96 | maximum |
| 110.0367 | −359 | −24 | −229 | // | 9 | 5 | 94 | 34 | 403 | 442 | ratios |
| 232.004 | −1034 | −173 | −2965 | −2 | // | 5 | 5318 | 69235 | 499 | 66 | |
| 175.0633 | −3 | −24 | −9 | −32 | −428 | // | 6 | 33 | 238 | 20 | |
| 254.1153 | −809 | −60 | −214 | −284 | −13865 | −2 | // | 125 | 536 | 136 | |
| 208.0737 | −15 | −51 | −17 | −199 | −164 | −52 | −10 | // | 8 | 20 | |
| 315.2409 | −1656 | −915 | −2602 | −524 | −984 | −4 | −551 | −30 | // | 272 | |
| 224.084 | −383 | −179 | −349 | −128 | −69 | −3 | −43 | −27 | −791 | // | |

Lower half = minimum ratios

TABLE B

| | 702.5682 | 189.0096 | 189.9936 | 110.0367 | 232.004 | 175.0633 | 254.1153 | 208.0737 | 315.2409 | 224.084 | |
|---|---|---|---|---|---|---|---|---|---|---|---|
| 702.5682 | // | 1218 | 468 | 257 | 341 | 15 | 88 | 11 | 240 | 393 | Upper |
| 189.0096 | −568 | // | 5372 | 24 | 4648 | 5 | 54 | 43 | 89 | 612 | half = |
| 189.9936 | −248 | −39 | // | 930 | 3527 | 2 | 77 | 13 | 266 | 96 | maximum |
| 110.0367 | −262 | −24 | −103 | // | 9 | 5 | 94 | 34 | 76 | 442 | ratios |
| 232.004 | −754 | −27 | −2760 | −2 | // | 5 | 5318 | 79 | 499 | 66 | |
| 175.0633 | −3 | −24 | −9 | −32 | −428 | // | 6 | 33 | 238 | 7 | |
| 254.1153 | −809 | −60 | −71 | −284 | −13865 | −2 | // | 125 | 536 | 136 | |
| 208.0737 | −15 | −51 | −14 | −199 | −164 | −52 | −10 | // | 8 | 17 | |
| 315.2409 | −220 | −601 | −671 | −303 | −984 | −3 | −179 | −30 | // | 272 | |
| 224.084 | −383 | −179 | −349 | −54 | −69 | −3 | −43 | −27 | −238 | // | |

Lower half = minimum ratios

TABLE C

| | 702.5682 | 189.0096 | 189.9936 | 110.0367 | 232.004 | 175.0633 | 254.1153 | 208.0737 | 315.2409 | 224.084 | |
|---|---|---|---|---|---|---|---|---|---|---|---|
| 702.5682 | // | 210 | 92 | 246 | 329 | 6 | 88 | 9 | 240 | 392 | Upper |
| 189.0096 | −566 | // | 101 | 24 | 10 | 5 | 20 | 24 | 49 | 27 | half = |
| 189.9936 | −220 | −2 | // | 56 | 68 | 2 | 77 | 7 | 20 | 16 | maximum |
| 110.0367 | −262 | −2 | −6 | // | 8 | 5 | 94 | 34 | 30 | 9 | ratios |
| 232.004 | −751 | −9 | −189 | −2 | // | 5 | 86 | 24 | 84 | 38 | |
| 175.0633 | −3 | −6 | −5 | −9 | −9 | // | 5 | 33 | 4 | 6 | |
| 254.1153 | −323 | −25 | −10 | −256 | −270 | −2 | // | 6 | 536 | 27 | |
| 208.0737 | −15 | −4 | −10 | −9 | −20 | −33 | −10 | // | 6 | 15 | |
| 315.2409 | −25 | −601 | −671 | −303 | −854 | −3 | −61 | −30 | // | 44 | |
| 224.084 | −30 | −33 | −49 | −25 | −40 | −2 | −2 | −25 | −69 | // | |

Lower half = minimum ratios

For each of Tables A, B, and C, the “lower half” refers to the section below the diagonal line indicated by the “//” signs and the “Upper half” refers to the section above that line.

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 189.0096 gram/mol, a compound having a mass of 224.084 gram/mol, and a compound having a mass of 254.1153 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 189.993 gram/mol, a compound having a mass of 224.084 gram/mol, a compound having a mass of 232.004 gram/mol and a compound having a mass of 254.115 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 175.0633 gram/mol, a compound having a mass of 208.0737 gram/mol and a compound having a mass of 254.1153 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 175.0633 gram/mol, a compound having a mass of 208.0737 gram/mol, a compound having a mass of 315.2409 gram/mol, and a compound having a mass of 702.5682 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 175.0633 gram/mol, and a compound having a mass of 315.2409 gram/mol, are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 175.0633 gram/mol, and a compound having a mass of 208.0737 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 175.0633 gram/mol, and a compound having a mass of 208.0737 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 208.0737 gram/mol, and a compound having a mass of 702.5682 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 175.0633 gram/mol, a compound having a mass of 208.0737 gram/mol, and a compound having a mass of 315.2409 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in (a) and analysed in (b).

In some preferred embodiments of the method for identifying a subject suffering from Alzheimer's dementia (AD) or being at risk of developing Alzheimer's dementia, at least the amounts of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in (a) and analysed in (b).

2^{nd} Aspect—Method for Identifying a Subject Suffering from Alzheimer's Dementia (AD) or being at Risk of Developing Alzheimer's Dementia (Modelling Based on Change)

In a second aspect, the invention relates to a method for identifying a subject suffering from Alzheimer's dementia (AD) or being at risk of developing Alzheimer's dementia, the method comprising:
(a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
(b) determining in a second body fluid sample of the subject the amount of the same at least one compound as in (a);
(c) determining the change of the amount of the at least one compound determined according to (a) compared to the respective amount of the at least one compound determined according to (b);
(d) analyzing the change as determined in (c) using a computer-implemented prediction algorithm that is capable of predicting AD in a subject based on the at least one compound, whereby a subject suffering from AD or being at risk of developing Alzheimer's dementia is identified if AD is predicted;
wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

The definitions indicated above in the section related to the first aspect apply also here in the section related to the second aspect of the invention.

Thiethylperazine (IUPAC: 2-(ethylsulfanyl)-10-[3-(4-methylpiperazin-1-yl) propyl]-10H-phenothiazine, CAS: 1420-55-9, TEP) has been proven effective in murine models of Alzheimer's disease (AD) to export amyloid beta (Abeta) from and through the endothelial cells of the blood-brain barrier to the periphery. It has also been shown that pharmaceutical activation of ABCC1 can reduce amyloid plaque deposition in the brain. The term “pharmaceutically acceptable”, as used herein, relates to a salt, which is pharmaceutically acceptable in the sense of being not deleterious to the recipient thereof and, preferably, being compatible with optional other ingredients of a formulation thereof. Preferably, a pharmaceutically acceptable salt is a salt of thiethylperazine causing at most moderate adverse drug reactions, preferably causing at most mild adverse drug reactions. As used herein, the term “mild” adverse reactions relates to adverse reactions not requiring medical intervention, such as skin rashes, headaches, digestive disturbances, fatigue, and the like; “moderate” adverse reactions are adverse reactions requiring medical intervention, but not being potentially life-threatening. Preferably, a pharmaceutically acceptable salt comprises one or more anion(s) selected from the group consisting of sulfate, pyrosulfate, bisulfate, sulfite, bisulfite, phosphate, monohydrogen phosphate, dihydrogen phosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, malate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenyl acetate, phenyl propionate, phenyl butyrate, citrate, lactate, β-hydroxybutyrate, glycolate, tartrate, methanesulfonate, propanesulfonate, naphthalene-1-sulfonate, naphthalene-2-sulfonate, almondate, adipate, alginate, mucate, aspartate, benzenesulfonate, butyrate, camphorate, camphorsulfonate, cyclopentane propionate, digluconate, dodecyl sulfate, ethane sulfonate, glucoheptanoate, glycerophosphate, hemisulfate, hexanoate, 2-hydroxy ethane sulfonate, 2-napthalene sulfonate, nicotinate, nitrate, palmitate, pectinate, persulfate, picrate, pivalate, salicylate, thiocyanate, tosylate, arginate, and undecanoate. More preferably, a pharmaceutically acceptable salt comprises one or 5 more anion(s) selected from the group consisting of chloride, sulfate, mesylate, besylate, tosylate, fumarate, malonate, malate, maleate, succinate, lactate, glycolate, citrate, aspartate and mandelate, preferably malate and/or maleate, more preferably maleate.

In some preferred embodiments of the method for identifying a subject suffering from Alzheimer's dementia (AD) or being at risk of developing Alzheimer's dementia of the second aspect, the at least one compound having a mass in gram/mol, of which the amount is determined in (a) and (b), the change is determined in (c) and analysed in (d) is selected from the group consisting of 110.0367, 232.004, 224.084, 254.1153, 189.9936, and 189.0096 (Group 2).

In some preferred embodiments of the method for identifying a subject suffering from Alzheimer's dementia (AD) or being at risk of developing Alzheimer's dementia of the second aspect, the at least one compound having a mass in gram/mol, of which the amount is determined in (a) and (b), the change is determined in (c) and analysed in (d) is at least a compound having a mass in gram/mol of 110.0367.

In some preferred embodiments of the method for identifying a subject suffering from Alzheimer's dementia (AD) or being at risk of developing Alzheimer's dementia of the second aspect, the amount of at least two compounds, preferably at least three compounds, more preferably at least four compounds, more preferably at least five compounds, having a mass in gram/mol selected from the group consisting of Group 1 or Group 2 is determined in (a) and (b), the change compared in (c) and analysed in (d), wherein more preferably one of the at least two compounds, preferably one of the at least three compounds, more preferably one of the at least four compounds, more preferably one of the at least five compounds is the compound having a mass in gram/mol of 110.0367.

In some preferred embodiments of the method for identifying a subject suffering from Alzheimer's dementia (AD) or being at risk of developing Alzheimer's dementia of the second aspect, the amount of at least two compounds, preferably at least six compounds or at least seven compounds, having a mass in gram/mol selected from the group consisting of Group 1 or Group 2 is determined in (a) and (b), the change compared in (c) and analysed in (d), wherein more preferably one of the at least two compounds, more preferably one of the at least six compounds or one of the at least seven compounds, is the compound having a mass in gram/mol of 110.0367.

In some preferred embodiments of the method for identifying a subject suffering from Alzheimer's dementia (AD) or being at risk of developing Alzheimer's dementia of the second aspect, the amount of at least one further compound having a mass in gram/mol selected from the group consisting of 208.0737, 315.2409, 175.0633, 702.5682, 161.0477, and 97.9769 (Group 3), preferably from the group consisting of 208.0737, 315.2409, 175.0633, 702.5682 (Group 4), is additionally determined in (a) and (b), the change compared in (c) and analysed in (d).

In some preferred embodiments of the method for identifying a subject suffering from Alzheimer's dementia (AD) or being at risk of developing Alzheimer's dementia of the second aspect, the method comprises:
(a) determining in a first body fluid sample of the subject the amount of at least two compounds having a mass in gram/mol selected from the group consisting of Group 3, preferably from the group consisting of Group 4;
(b) determining in a second body fluid sample of the subject the amount of the same at least two compounds as in (a);
(c) determining the change of the amount of the at least two compounds determined according to (a) compared to the respective amount of the at least two compounds determined according to (b);
(d) analyzing the changes as determined in (c) using a computer-implemented prediction algorithm that is capable of predicting AD in a subject based on the at least one compound, whereby a subject suffering from AD or being at risk of developing Alzheimer's dementia is identified if AD is predicted.

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 189.0096 gram/mol, a compound having a mass of 224.084 gram/mol, and a compound having a mass of 254.1153 gram/mol is determined in (a) and (b), the change compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 189.993 gram/mol, a compound having a mass of 224.084 gram/mol, a compound having a mass of 232.004 gram/mol and a compound having a mass of 254.115 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 175.0633 gram/mol, a compound having a mass of 208.0737 gram/mol and a compound having a mass of 254.1153 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 175.0633 gram/mol, a compound having a mass of 208.0737 gram/mol, a compound having a mass of 315.2409 gram/mol, and a compound having a mass of 702.5682 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 175.0633 gram/mol, and a compound having a mass of 315.2409 gram/mol, is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 208.0737 gram/mol, a compound having a mass of 232.004 gram/mol, and a compound having a mass of 315.2409 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 175.0633 gram/mol, and a compound having a mass of 208.0737 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 175.0633 gram/mol, and a compound having a mass of 208.0737 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 208.0737 gram/mol, and a compound having a mass of 702.5682 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 175.0633 gram/mol, a compound having a mass of 208.0737 gram/mol, and a compound having a mass of 315.2409 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol is determined in (a) and (b), the changes compared in (c) and analysed in (d).

In some preferred embodiments of the method for identifying a subject suffering from Alzheimer's dementia (AD) or being at risk of developing Alzheimer's dementia of the second aspect, at least the amount of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol is determined in (a) and (b), the changes compared in (c) and analysed in (d).

The amount determination in any one of steps (a) and/or (b) is done by mass spectrometry (MS), preferably by a tandem mass spectrometry (MS/MS), more preferably by a tandem mass spectrometry in combination with a preceding liquid chromatography (LC-MS/MS); further details can be found in the section related to the first aspect of the invention. The method is an in vitro method. The term “subject”, as used herein, relates to a multicellular animal, preferably to a vertebrate, more preferably to a mammal, more preferably a human. The body fluid sample is selected from the group consisting of blood sample, urine sample, liquor sample (CSF) and mixtures of two or more thereof, preferably the body fluid sample is a blood sample; and respectively the first body fluid sample and the second body fluid sample are independently of each other selected from the group consisting of blood sample, urine sample, liquor sample (CSF) and mixtures of two or more thereof, wherein preferably, the first body fluid sample and the second body fluid sample are the same kind of sample and are both selected from the group consisting of blood sample, urine sample, liquor sample (CSF) and mixtures of two or more thereof, wherein more preferably, first body fluid sample and second body fluid sample are both each a blood sample (first blood sample and second blood sample).

3^{rd} Aspect—Method for Identifying a Subject Suffering from Alzheimer's Dementia (AD) or being at Risk of Developing Alzheimer's Dementia (Comparison of Amounts Before and After TEP Administration)

In a third aspect, the invention relates to a method for identifying a subject suffering from Alzheimer's dementia (AD) or being at risk of developing Alzheimer's dementia, the method comprising:
(a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
(b) determining in a second body fluid sample of the subject the amount of the same at least one compound as in (a);
(c) comparing the amount of the at least one compound determined according to (a) to the respective amount of the at least one compound determined according to (b), wherein a change of the amount of the at least one compound according to (b) compared with the amount as determined in (a) indicates Alzheimer's dementia for the subject or the risk of developing Alzheimer's dementia for the subject;
   wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

The definitions indicated above in the section related to the first aspect apply also here in the section related to the third aspect of the invention. Thiethylperazine (IUPAC: 2-(ethylsulfanyl)-10-[3-(4-methylpiperazin-1-yl) propyl]-10H-phenothiazine, CAS: 1420-55-9, TEP) and pharmaceutically acceptable salt thereof are also as defined above in the section related to the second aspect.

In some preferred embodiments of the method for identifying a subject suffering from Alzheimer's dementia (AD) or being at risk of developing Alzheimer's dementia of the third aspect, the at least one compound having a mass in gram/mol is selected from the group consisting of 110.0367, 232.004, 224.084, 254.1153, 189.9936, and 189.0096 (Group 2).

In some preferred embodiments of the method for identifying a subject suffering from Alzheimer's dementia (AD) or being at risk of developing Alzheimer's dementia of the third aspect, in (a) and (b) at least the amount of a compound having a mass of 110.0367 gram/mol is determined and compared in (c).

In some preferred embodiments of the method for identifying a subject suffering from Alzheimer's dementia (AD) or being at risk of developing Alzheimer's dementia of the third aspect, the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in steps (a) and (b) and compared in (c), wherein a change of the amount of each of the at least two compounds, preferably each of the at least three compounds, more preferably each of the at least four compounds, more preferably each of the at least five compounds, according to (b) compared with the amount as determined in (a) indicates Alzheimer's dementia for the subject or the risk of developing Alzheimer's dementia for the subject, wherein more preferably one of the at least two compounds, preferably one of the at least three compounds, more preferably one of the at least four compounds, more preferably one of the at least five compounds is the compound having a mass of 110.0367 gram/mol.

In some preferred embodiments of the method for identifying a subject suffering from Alzheimer's dementia (AD) or being at risk of developing Alzheimer's dementia of the third aspect, the amount of at least two compounds, preferably at least six compounds or at least seven compounds, having a mass in gram/mol selected from the group consisting of Group 1 or Group 2 is determined in a first body fluid sample of the subject in steps (a) and (b) and compared in (c), wherein a change of the amount of each of the at least two compounds, preferably each of the at least six compounds, more preferably each of the at least seven compounds, according to (b) compared with the amount as determined in (a) indicates Alzheimer's dementia for the subject or the risk of developing Alzheimer's dementia for the subject, wherein more preferably one of the at least two compounds, more preferably one of the at least six compounds or one of the at least seven compounds, is the compound having a mass of 110.0367 gram/mol.

In some preferred embodiments of the method for identifying a subject suffering from Alzheimer's dementia (AD) or being at risk of developing Alzheimer's dementia of the third aspect, the amount of at least one further compound having a mass in gram/mol selected from the group consisting of 208.0737, 315.2409, 175.0633, 702.5682, 161.0477, and 97.9769 (Group 3), more preferably from the group consisting of 208.0737, 315.2409, 175.0633, 702.5682 (Group 4), is additionally determined in a first body fluid sample of the subject in steps (a) and (b) and compared in (c), wherein a change of the amount of the at least one compound according to (b) compared with the amount as determined in (a) and a change of the amount of the at least one further compound according to (b) compared with the amount as determined in (a) indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, the method comprises:
(a) determining in a first body fluid sample of the subject the amount of at least two compounds having a mass in gram/mol selected from the group consisting of Group 3, preferably Group 4;
(b) determining in a second body fluid sample of the subject the amount of the same at least two compound as in (a);
(c) comparing the amounts of the at least two compounds determined according to (a) to the respective amounts of the at least two compounds determined according to (b), wherein a change of the amounts of the at least two compounds according to (b) compared with the amount as determined in (a) indicates Alzheimer's dementia for the subject or the risk of developing Alzheimer's dementia for the subject;
wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 189.0096 gram/mol, a compound having a mass of 224.084 gram/mol, and a compound having mass of 254.1153 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 189.993 gram/mol, a compound having a mass of 224.084 gram/mol, a compound having a mass of 232.004 gram/mol and a compound having a mass of 254.115 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 175.0633 gram/mol, a compound having a mass of 208.0737 gram/mol and a compound having a mass of 254.1153 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 175.0633 gram/mol, a mass of 208.0737 gram/mol, a compound having a mass of 315.2409 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 175.0633 gram/mol, and a compound having a mass of 315.2409 gram/mol, are determined in a first body fluid sample of the subject in step (a) and (b) and compared in (c), wherein a change of the amounts of each of these compounds according to (b) compared with the amounts as determined in (a) by at least 2% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 175.0633 gram/mol, and a compound having a mass of 208.0737 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 175.0633 gram/mol, and a compound having a mass of 208.0737 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 208.0737 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 175.0633 gram/mol, a compound having a mass of 208.0737 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 40% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.

In some preferred embodiments of the method for identifying a subject suffering from Alzheimer's dementia (AD) or being at risk of developing Alzheimer's dementia of the third aspect, at least the amounts of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in a first body fluid sample of the subject in step (a) and (b) and compared in (c), wherein a change of the amounts of each of these compounds according to (b) compared with the amounts as determined in (a) by at least 2% indicates Alzheimer's dementia for the subject or the risk of developing Alzheimer's dementia for the subject.

The amount determination in any one of steps (a) and/or (b) is done by mass spectrometry (MS), preferably by a tandem mass spectrometry (MS/MS), more preferably by a tandem mass spectrometry in combination with a preceding liquid chromatography (LC-MS/MS) further details can be found in the section related to the first aspect of the invention. The method is an in vitro method. The term “subject”, as used herein, relates to a multicellular animal, preferably to a vertebrate, more preferably to a mammal, more preferably a human. The body fluid sample is selected from the group consisting of blood sample, urine sample, liquor sample (CSF) and mixtures of two or more thereof, preferably the body fluid sample is a blood sample; and respectively the first body fluid sample and the second body fluid sample are independently of each other selected from the group consisting of blood sample, urine sample, liquor sample (CSF) and mixtures of two or more thereof, wherein preferably, the first body fluid sample and the second body fluid sample are the same kind of sample and are both selected from the group consisting of blood sample, urine sample, liquor sample (CSF) and mixtures of two or more thereof, wherein more preferably, first body fluid sample and second body fluid sample are both each a blood sample (first blood sample and second blood sample).

4^{th} Aspect—Method for Identifying a Subject Suffering from Alzheimer's Dementia (AD) (Comparison of Amount after TEP Administration with Reference Value)

A fourth aspect of the invention refers to a method for identifying a subject suffering from Alzheimer's dementia (AD), the method comprising:
(a) determining in a body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
(b) comparing the amount of the at least one compound determined according to (a) to a mean amount of a healthy subject, wherein a change of the amount of the at least one compound according to (a) compared with the mean amount of a healthy subject, preferably by at least 3% based on the mean amount of a healthy subject being 100%, indicates Alzheimer's dementia for the subject or indicates that the subject is at risk of developing Alzheimer's dementia;
wherein said body fluid sample had been obtained after the subject had been administered thiethylperazine or a pharmaceutically acceptable salt thereof.

The definitions given in the section related to the first aspect and the explanations related to thiethylperazine (TEP) and its pharmaceutically acceptable salts given in the second and third section apply also here in the section related to the fourth aspect of the invention.

In some preferred embodiments related to the method for identifying a subject suffering from Alzheimer's dementia (AD) or being at risk of developing Alzheimer's dementia of the fourth aspect, the least one compound having a mass in gram/mol is selected from the group consisting of 110.0367, 232.004, 224.084, 254.1153, 189.9936, and 189.0096 (Group 2).

In some preferred embodiments related to the method for identifying a subject suffering from Alzheimer's dementia (AD) or being at risk of developing Alzheimer's dementia of the fourth aspect, in (a) at least the amount of a compound having a mass in gram/mol of 110.0367 is determinedand compared in (b).

In some preferred embodiments related to the method for identifying a subject suffering from Alzheimer's dementia (AD) or being at risk of developing Alzheimer's dementia of the fourth aspect, the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in step (a) and compared in (b), wherein a change of the amount of each of the at least two compounds, preferably each of the at least three compounds, more preferably each of the at least four compounds, more preferably of the at least five compounds, indicates Alzheimer's dementia for the subject or the risk of developing Alzheimer's dementia for the subject, wherein more preferably one of the at least two compounds, preferably one of the at least three compounds, more preferably one of the at least four compounds, more preferably one of the at least five compounds is the compound having a mass in gram/mol of 110.0367.

In some preferred embodiments related to the method for identifying a subject suffering from Alzheimer's dementia (AD) or being at risk of developing Alzheimer's dementia of the fourth aspect, the amount of at least two compounds, preferably at least six compounds, more preferably at least seven compounds, having a mass in gram/mol selected from the group consisting of Group 1 or Group 2 is determined in a first body fluid sample of the subject in step (a) and compared in (b), wherein a change of the amount of each of the at least two compounds, preferably each of the at least six compounds, more preferably each of the at least seven compounds, indicates Alzheimer's dementia for the subject or the risk of developing Alzheimer's dementia for the subject, wherein more preferably one of the at least two compounds, more preferably one of the at least six compounds or one of the at least seven compounds, is the compound having a mass in gram/mol of 110.0367.

In some preferred embodiments related to the method for identifying a subject suffering from Alzheimer's dementia (AD) or being at risk of developing Alzheimer's dementia of the fourth aspect, the amount of at least one further compound having a mass in gram/mol selected from the group consisting of 208.0737, 315.2409, 175.0633, 702.5682, 161.0477, and 97.9769 (Group 3), more preferably from the group consisting of 208.0737, 315.2409, 175.0633, 702.5682 (Group 4), is determined in a first body fluid sample of the subject in steps (a) and compared in (b), wherein a change of the amount of each of the at least one compound compared with the mean amount of a healthy subject, preferably by at least 3% based on the mean amount of a healthy subject being 100% indicates Alzheimer's dementia for the subject or the risk of developing Alzheimer's dementia for the subject.

The fourth aspect of the invention also relates to a method for identifying a subject suffering from Alzheimer's dementia (AD) or being at risk of developing Alzheimer's dementia, the method comprising:
(a) determining in a body fluid sample of the subject the amounts of at least two compounds having a mass in gram/mol selected from the group consisting of Group 3, preferably of Group 4;
(b) comparing the amounts of the at least two compounds determined according to (a) to a mean amount of a healthy subject, wherein a change of the amounts of the at least two compounds according to (a) compared with the mean amount of a healthy subject, preferably by at least 3% based on the mean amount of a healthy subject being 100%, indicates Alzheimer's dementia for the subject or indicates that the subject is at risk of developing Alzheimer's dementia;
wherein said body fluid sample had been obtained after the subject had been administered thiethylperazine or a pharmaceutically acceptable salt thereof.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 189.0096 gram/mol, a compound having a mass of 224.084 gram/mol, and a compound having a mass of 254.1153 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 189.993 gram/mol, a compound having a mass of 224.084 gram/mol, a compound having a mass of 232.004 gram/mol and a compound having a mass of 254.115 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 175.0633 gram/mol, a compound having a mass of 208.0737 gram/mol and a compound having a mass of 254.1153 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 175.0633 gram/mol, a compound having a mass of 208.0737 gram/mol, a compound having a mass of 315.2409 gram/mol, and a compound having a mass of 702.5682 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 175.0633 gram/mol, and a compound having a mass of 315.2409 gram/mol, are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 175.0633 gram/mol, and a compound having a mass of 208.0737 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 175.0633 gram/mol, and a compound having a mass of 208.0737 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 208.0737 gram/mol, and a compound having a mass of 702.5682 gram/mol are determined in a first body fluid sample of the subject in step (a) and compared in (b), wherein a change of the amounts of each of these compounds by at least 12% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 175.0633 gram/mol, a compound having a mass of 208.0737 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, at least the amounts of a compound having a mass 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in a first body fluid sample of the subject in step (a) and compared in (b), wherein a change of the amounts of each of these compounds a by at least 3% indicates Alzheimer's dementia for the subject or the risk of developing Alzheimer's dementia for the subject.

In some preferred embodiments related to the method for identifying a subject suffering from Alzheimer's dementia (AD) or being at risk of developing Alzheimer's dementia of the fourth aspect, the mean amount of a healthy subject is a reference value of a healthy subject from literature, or a reference value previously determined from healthy subjects or a reference value from a body fluid sample of the subject prior to TEP administration.

The amount determination in any one of steps (a) and/or (b) is done by mass spectrometry (MS), preferably by a tandem mass spectrometry (MS/MS), more preferably by a tandem mass spectrometry in combination with a preceding liquid chromatography (LC-MS/MS) further details can be found in the section related to the first aspect of the invention. The method is an in vitro method. The term “subject”, as used herein, relates to a multicellular animal, preferably to a vertebrate, more preferably to a mammal, more preferably a human. The body fluid sample is selected from the group consisting of blood sample, urine sample, liquor sample (CSF) and mixtures of two or more thereof, preferably the body fluid sample is a blood sample; and respectively the first body fluid sample and the second body fluid sample are independently of each other selected from the group consisting of blood sample, urine sample, liquor sample (CSF) and mixtures of two or more thereof, wherein preferably, the first body fluid sample and the second body fluid sample are the same kind of sample and are both selected from the group consisting of blood sample, urine sample, liquor sample (CSF) and mixtures of two or more thereof, wherein more preferably, first body fluid sample and second body fluid sample are both each a blood sample (first blood sample and second blood sample).

5^{th} Aspect—2^{nd} Diagnostic Uses

A fifth aspect of the invention relates to at least one compound having a mass in gram/mol selected from the group consisting of Group 1, or Group 2, or as defined in any one of the preferred embodiments described in the previous sections related to the first, the second, the third or the fourth aspect of the invention, and optionally in combination with at least one compound having a mass in gram/mol selected from the group consisting of Group 3, preferably of Group 4, for diagnosing AD in a subject or for determining the risk of a subject to develop AD

Groups 1, 2, 3 and 4 are as defined above in the sections related to the first, the second, the third or the fourth aspect of the invention.

In some preferred embodiments of the at least one compound for use according to the fifth aspect, at least two compounds having a mass in gram/mol selected from the group consisting of Group 1, or Group 2 described above in the sections related to the first, the second, the third or the fourth aspect of the invention, preferably at least three compounds having a mass in gram/mol selected from the group consisting of Group 1, or Group 2 as defined in any one of the preferred embodiments described above in the sections related to the first, the second, the third or the fourth aspect of the invention, more preferably at least four compounds having a mass in gram/mol selected from the group consisting of Group 1, or Group 2 as defined in any one of the preferred embodiments described above in the sections related to the first, the second, the third or the fourth aspect of the invention; more preferably at least five compounds having a mass in gram/mol selected from the group consisting of Group 1, or Group 2 as defined in any one of the preferred embodiments described above in the sections related to the first, the second, the third or the fourth aspect of the invention, each optionally in combination with at least one compound having a mass in gram/mol selected from the group consisting of Group 3 or Group 4, are used.

In some preferred embodiments of the at least one compound for use according to the fifth aspect, at least two compounds having a mass in gram/mol selected from the group consisting of Group 1, or Group 2 as defined in any one of the preferred embodiments described above in the sections related to the first, the second, the third or the fourth aspect of the invention, preferably at least six compounds having a mass in gram/mol selected from the group consisting of Group 1, or Group 2 as defined in any one of the preferred embodiments described above in the sections related to the first, the second, the third or the fourth aspect of the invention, more preferably at least seven compounds having a mass in gram/mol selected from the group consisting of Group 1, or Group 2 a as defined in any one of the preferred embodiments described above in the sections related to the first, the second, the third or the fourth aspect of the invention; each optionally in combination with at least one compound having a mass in gram/mol selected from the group consisting of Group 3 or Group 4, are used.

In some preferred embodiments of the at least one compound for use according to the fifth aspect, at least two compounds having a mass in gram/mol selected from the group consisting of Group 3 or Group 4 for diagnosing AD in a subject or for determining the risk of a subject to develop AD.

### 2^{nd} Diagnostic Use (Alternative Wording)

The fifth aspect of the invention also relates to the use of at least one compound having a mass in gram/mol selected from the group consisting of Group 1, or Group 2 as defined in any one of the preferred embodiments described above in the sections related to the first, the second, the third or the fourth aspect of the invention, and optionally in combination with at least one compound having a mass in gram/mol selected from the group consisting of Group 3 or Group 4 in a sample of a subject for diagnosing AD in said subject or for determining the risk of said subject to develop AD.

Groups 1, 2, 3 and 4 are as defined above in the sections related to the first, the second, the third or the fourth aspect of the invention.

In some preferred embodiments of the use according to the fifth aspect, at least two, preferably at least three, more preferably at least four, more preferably at least five, compounds having a mass in gram/mol selected from the group consisting of Group 1, or Group 2 a as defined in any one of the preferred embodiments described above in the sections related to the first, the second, the third or the fourth aspect of the invention and optionally in combination with at least one compound having a mass in gram/mol selected from the group consisting of Group 3 or Group 4 in a sample of a subject are used for diagnosing AD in said subject or for determining the risk of said subject to develop AD.

In some preferred embodiments of the use according to the fifth aspect, at least two, preferably at least six, more preferably at least seven, compounds having a mass in gram/mol selected from the group consisting of Group 1, or Group 2 as defined in any one of the preferred embodiments described above in the sections related to the first, the second, the third or the fourth aspect of the invention and optionally in combination with at least one compound having a mass in gram/mol selected from the group consisting of Group 3 or Group 4 in a sample of a subject are used for diagnosing AD in said subject or for determining the risk of said subject to develop AD.

In some preferred embodiments of the use according to the fifth aspect, at least two compounds having a mass in gram/mol selected from the group consisting of Group 3 or group 4 in a sample of a subject for diagnosing AD in said subject or for determining the risk of said subject to develop AD.

### 6^{th} Aspect—Device

A sixth aspect of the invention relates to a device adapted for identifying a subject suffering from Alzheimer's dementia (AD) or being at risk of developing Alzheimer's dementia, the device comprising:
(a) an analyzing unit capable of determining in a first body fluid sample of the subject the amount of at least one compound having a mass in gram/mol selected from the group consisting of Group 1;
(b) a data processor adopted for analyzing the determined amount for the at least one compound using a prediction algorithm implemented in said data processor that is capable of predicting AD in a subject based on the at least two compounds, whereby a subject suffering from AD is identified if AD is predicted or a subject being at risk of developing Alzheimer's dementia is identified if AD is predicted.

Preferred embodiments of the device, especially related to compounds having specific masses in gram/mol and specific groups thereof such as Group 2, Group 3 and Group 4, are as described above in the sections related to the first, the second, the third or the fourth aspect of the invention. Also the definitions indicated above in these sections apply also here in the section related to the device.

### 7^{th} Aspect—Kit

In a seventh aspect, the invention relates to a kit for identifying a subject suffering from Alzheimer's dementia (AD) or being at risk of developing Alzheimer's dementia, the kit comprising a reference sample of at least one compound having a mass in gram/mol selected from the group consisting of Group 1 or Group 2, optionally a reference sample of at least one compound having a mass in gram/mol selected from the group consisting of Group 3, or optionally a reference sample of at least one compound having a mass in gram/mol selected from the group consisting of Group 4, and optionally one or more reagents for sample preparation and/or extraction.

Preferred embodiments of the device, especially related to compounds having specific masses in gram/mol and specific groups thereof such as Group 2, Group 3 and Group 4, are as described above in the sections related to the first, the second, the third or the fourth aspect of the invention. Also the definitions indicated above in these sections apply also here in the section related to the device.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”.
1 A method for identifying a subject suffering from Alzheimer's dementia (AD) or being at risk of developing Alzheimer's dementia, the method comprising:
   (a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
   (b) analyzing the determined amount for the at least one compound using a computer-implemented prediction algorithm that is capable of predicting AD in a subject based on the at least one compound, whereby a subject suffering from AD or being at risk of developing Alzheimer's dementia is identified if AD is predicted,
   wherein preferably, the first body fluid sample derives from a subject after the subject had been administered thiethylperazine (TEP) or a pharmaceutically acceptable salt thereof, wherein the sample was preferably taken within 0.5 hours to 8 hours after TEP administration.
2. The method for identifying a subject suffering from Alzheimer's dementia (AD) or being at risk of developing Alzheimer's dementia according to embodiment 1, wherein the least one compound having a mass in gram/mol is selected from the group consisting of 110.0367, 232.004, 224.084, 254.1153, 189.9936, and 189.0096 (Group 2).
3. The method for identifying a subject suffering from Alzheimer's dementia (AD) or being at risk of developing Alzheimer's dementia according to embodiment 1 or 2, wherein at least a compound having a mass in gram/mol of 110.0367 is determined in (a) and the determined amount thereof is analysed in (b).
4. The method for identifying a subject suffering from Alzheimer's dementia (AD) or being at risk of developing Alzheimer's dementia according to any one of embodiments 1 to 3, wherein the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in step (a) and analysed in (b), wherein more preferably one of the at least two compounds, preferably one of the at least three compounds, more preferably one of the at least four compounds, more preferably one of the at least five compounds is the compound having a mass in gram/mol of 110.0367.
5. The method for identifying a subject suffering from Alzheimer's dementia (AD) or being at risk of developing Alzheimer's dementia according to any one of embodiments 1 to 4, wherein the amount of at least two compounds, preferably at least six compounds, more preferably at least seven compounds, having a mass in gram/mol selected from the group consisting of Group 1 or Group 2 is determined in a first body fluid sample of the subject in step (a) and analysed in (b), wherein more preferably one of the at least two compounds, more preferably one of the at least six compounds or one of the at least seven compounds, is the compound having a mass in gram/mol of 110.0367.
6. The method for identifying a subject suffering from Alzheimer's dementia (AD) or being at risk of developing Alzheimer's dementia according to any one of embodiments 1 to 4, wherein the amount of at least one further compound having a mass in gram/mol selected from the group consisting of 208.0737, 315.2409, 175.0633, 702.5682, 161.0477, and 97.9769 (Group 3), preferably from the group consisting of 208.0737, 315.2409, 175.0633, 702.5682 (Group 4), is additionally determined in a first body fluid sample of the subject in steps (a) and additionally analysed in (b).
7. A method for identifying a subject suffering from Alzheimer's dementia (AD) or being at risk of developing Alzheimer's dementia, the method comprising:
   (a) determining in a first body fluid sample of the subject the amounts of at least two compounds having a mass in gram/mol selected from the group consisting of Group 3, preferably of Group 4;
   (b) analyzing the determined amounts for the at least two compounds using a computer-implemented prediction algorithm that is capable of predicting AD in a subject based on the at least two compounds, whereby a subject suffering from AD or being at risk of developing Alzheimer's dementia is identified if AD is predicted.
8. The method for identifying a subject suffering from Alzheimer's dementia (AD) or being at risk of developing Alzheimer's dementia according to any one of embodiments 1 to 7, wherein said computer-implemented prediction algorithm uses a supervised machine-learning algorithm that has been trained by a training dataset comprising quantitative or qualitative data for
   the at least one, preferably at least two, more preferably at least three, more preferably at least four, more preferably at least five, compound(s) from Group 1, or Group 2, optionally in combination with the at least one further compound from Group 3; or optionally in combination with the at least one further compound from Group 4, or
   the at least two, more preferably at least six, more preferably at least seven, compound(s) from Group 1, or Group 2 optionally in combination with the at least one further compound from Group 3, or optionally in combination with the at least one further compound from Group 4; or
   the at least two compounds from Group 3, preferably from Group 4;
in body fluid samples from subjects known to suffer from AD and subjects known not to suffer from AD.
9. The method of embodiment 8, wherein said supervised machine-learning algorithm establishes a classifying algorithm selected from the group consisting of Logistic Regression, stochastic gradient descent classifier (SGDClassifier), Decision Tree classifier, Random Tree classifier, Extra Tree classifier, AdaBoost Classifier, Multi-layer Perceptron classifier, Gradient Boosting Classifier, Gaussian Naïve Bayes, K Nearest Neighbor classifier, C-Support Vector Classification, Quadratic Discriminant Analysis, and Linear Regression.
10. The method of embodiment 9, wherein said supervised machine-learning algorithm establishes and/or applies a logistic regression model said logistic regression model being based on equitation (I)

y=I+C₁*F₁+C₂*F₂+ . . . +Cₙ*Fₙ, (I)
wherein I is the Intercept and Cₙ the coefficient of Mass Fₙ.
12. The method according to any one of embodiments 1 to 10, wherein at least the amounts of a compound having a mass of 110.0367 gram/mol, a compound having a mass of 189.0096 gram/mol, a compound having a mass of 224.084 gram/mol, and a compound having a mass of 254.1153 gram/mol are determined in (a) and analysed in (b).
13. The method according to any one of embodiments 1 to 10, wherein at least the amounts of a compound having a mass of 189.993 gram/mol, a compound having a mass of 224.084 gram/mol, a compound having a mass of 232.004 gram/mol and a compound having a mass of 254.115 gram/mol are determined in (a) and analysed in (b).
14. The method according to any one of embodiments 1 to 10, wherein at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in (a) and analysed in (b).
15. The method according to any one of embodiments 1 to 10, wherein at least the amounts of a compound having a mass of 175.0633 gram/mol, a compound having a mass of 208.0737 gram/mol and a compound having a mass of 254.1153 gram/mol are determined in (a) and analysed in (b).
16. The method according to any one of embodiments 1 to 10, wherein at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in (a) and analysed in (b).
17. The method according to any one of embodiments 1 to 10, wherein at least the amounts of a compound having a mass of 175.0633 gram/mol, a compound having a mass of 208.0737 gram/mol, a compound having a mass of 315.2409 gram/mol, and a compound having a mass of 702.5682 gram/mol are determined in (a) and analysed in (b).
18. The method according to any one of embodiments 1 to 10, wherein at least the amounts of a compound having a mass of 110.0367 gram/mol, a compound having a mass of 175.0633 gram/mol, and a compound having a mass of 315.2409 gram/mol, are determined in (a) and analysed in (b).
19. The method according to any one of embodiments 1 to 10, wherein at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in (a) and analysed in (b).
20. The method according to any one of embodiments 1 to 10, wherein at least the amounts of a compound having a mass of 175.0633 gram/mol, and a compound having a mass of 208.0737 gram/mol are determined in (a) and analysed in (b).
21. The method according to any one of embodiments 1 to 10, wherein at least the amounts of a compound having a mass of 110.0367 gram/mol, a compound having a mass of 175.0633 gram/mol, and a compound having a mass of 208.0737 gram/mol are determined in (a) and analysed in (b).
22. The method according to any one of embodiments 1 to 10, wherein at least the amounts of a compound having a mass of 208.0737 gram/mol, and a compound having a mass of 702.5682 gram/mol are determined in (a) and analysed in (b).
23. The method according to any one of embodiments 1 to 10, wherein at least the amounts of a compound having a mass of 175.0633 gram/mol, a compound having a mass of 208.0737 gram/mol, and a compound having a mass of 315.2409 gram/mol are determined in (a) and analysed in (b).
24. The method according to any one of embodiments 1 to 10, wherein at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in (a) and analysed in (b).
25. The method according to any one of embodiments 1 to 10, wherein at least the amounts of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in (a) and analysed in (b).
25. A method for identifying a subject suffering from Alzheimer's dementia (AD) or being at risk of developing Alzheimer's dementia, the method comprising:
   (a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
   (b) determining in a second body fluid sample of the subject the amount of the same at least one compound as in (a);
   (c) determining the change of the amount of the at least one compound determined according to (a) compared to the respective amount of the at least one compound determined according to (b);
   (d) analyzing the change as determined in (c) using a computer-implemented prediction algorithm that is capable of predicting AD in a subject based on the at least one compound, whereby a subject suffering from AD or being at risk of developing Alzheimer's dementia is identified if AD is predicted;
   wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.
26. The method according to embodiment 25, wherein the at least one compound having a mass in gram/mol, of which the amount is determined in (a) and (b), the change is determined in (c) and analysed in (c) is selected from the group consisting of 110.0367, 232.004, 224.084, 254.1153, 189.9936, and 189.0096 (Group 2).
27. The method according to embodiment 25 or 26, wherein the at least one compound having a mass in gram/mol, of which the amount is determined in (a) and (b), the change is determined in (c) and analysed in (c) is at least a compound having a mass in gram/mol of 110.0367.
28. The method according to any one of embodiments 25 to 27, wherein the amount of at least two compounds, preferably at least three compounds, more preferably at least four compounds, more preferably at least five compounds, having a mass in gram/mol selected from the group consisting of Group 1 or Group 2 is determined in (a) and (b), the change compared in (c) and analysed in (d), wherein more preferably one of the at least two compounds, preferably one of the at least three compounds, more preferably one of the at least four compounds, more preferably one of the at least five compounds is the compound having a mass in gram/mol of 110.0367.
29. The method according to any one of embodiments 25 to 28, wherein the amount of at least two compounds, preferably at least six compounds or at least seven compounds, having a mass in gram/mol selected from the group consisting of Group 1 or Group 2 is determined in (a) and (b), the change compared in (c) and analysed in (d), wherein more preferably one of the at least two compounds, more preferably one of the at least six compounds or one of the at least seven compounds, is the compound having a mass in gram/mol of 110.0367.
30. The method according to any one of embodiments 25 to 29, wherein the amount of at least one further compound having a mass in gram/mol selected from the group consisting of 208.0737, 315.2409, 175.0633, 702.5682, 161.0477, and 97.9769 (Group 3), preferably from the group consisting of 208.0737, 315.2409, 175.0633, 702.5682 (Group 4), is additionally determined in (a) and (b), the change compared in (c) and analysed in (d).
31. A method for identifying a subject suffering from Alzheimer's dementia (AD) or being at risk of developing Alzheimer's dementia, the method comprising:
   (a) determining in a first body fluid sample of the subject the amount of at least two compounds having a mass in gram/mol selected from the group consisting of Group 3, preferably from the group consisting of Group 4;
   (b) determining in a second body fluid sample of the subject the amount of the same at least two compounds as in (a);
   (c) determining the change of the amount of the at least two compounds determined according to (a) compared to the respective amount of the at least two compounds determined according to (b);
   (d) analyzing the changes as determined in (c) using a computer-implemented prediction algorithm that is capable of predicting AD in a subject based on the at least one compound, whereby a subject suffering from AD or being at risk of developing Alzheimer's dementia is identified if AD is predicted.
32. The method according to any one of embodiments 25 to 30, wherein at least the amount of a compound having a mass of 110.0367 gram/mol, a compound having a mass of 189.0096 gram/mol, a compound having a mass of 224.084 gram/mol, and a compound having a mass of 254.1153 gram/mol is determined in (a) and (b), the change compared in (c) and analysed in (d).
33. The method according to any one of embodiments 25 to 30, wherein at least the amount of a compound having a mass of 189.993 gram/mol, a compound having a mass of 224.084 gram/mol, a compound having a mass of 232.004 gram/mol and a compound having a mass of 254.115 gram/mol is determined in (a) and (b), the changes compared in (c) and analysed in (d).
34. The method according to any one of embodiments 25 to 30, wherein at least the amount of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol is determined in (a) and (b), the changes compared in (c) and analysed in (d).
35. The method according to any one of embodiments 25 to 30, wherein at least the amount of a compound having a mass of 175.0633 gram/mol, a compound having a mass of 208.0737 gram/mol and a compound having a mass of 254.1153 gram/mol is determined in (a) and (b), the changes compared in (c) and analysed in (d).
36. The method according to any one of embodiments 25 to 30, wherein at least the amount of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol is determined in (a) and (b), the changes compared in (c) and analysed in (d).
37. The method according to any one of embodiments 25 to 30, wherein at least the amount of a compound having a mass of 175.0633 gram/mol, a compound having a mass of 208.0737 gram/mol, a compound having a mass of 315.2409 gram/mol, and a compound having a mass of 702.5682 gram/mol is determined in (a) and (b), the changes compared in (c) and analysed in (d).
38. The method according to any one of embodiments 25 to 30, wherein at least the amount of a compound having a mass of 110.0367 gram/mol, a compound having a mass of 175.0633 gram/mol, and a compound having a mass of 315.2409 gram/mol, is determined in (a) and (b), the changes compared in (c) and analysed in (d).
39. The method according to any one of embodiments 25 to 30, wherein at least the amount of a compound having a mass of 110.0367 gram/mol, a compound having a mass of 208.0737 gram/mol, a compound having a mass of 232.004 gram/mol, and a compound having a mass of 315.2409 gram/mol is determined in (a) and (b), the changes compared in (c) and analysed in (d).
40. The method according to any one of embodiments 25 to 30, wherein at least the amount of a compound having a mass of 175.0633 gram/mol, and a compound having a mass of 208.0737 gram/mol is determined in (a) and (b), the changes compared in (c) and analysed in (d).
41. The method according to any one of embodiments 25 to 30, wherein at least the amount of a compound having a mass of 110.0367 gram/mol, a compound having a mass of 175.0633 gram/mol, and a compound having a mass of 208.0737 gram/mol is determined in (a) and (b), the changes compared in (c) and analysed in (d).
42. The method according to any one of embodiments 25 to 30, wherein at least the amount of a compound having a mass of 208.0737 gram/mol, and a compound having a mass of 702.5682 gram/mol is determined in (a) and (b), the changes compared in (c) and analysed in (d).
43. The method according to any one of embodiments 25 to 30, wherein at least the amount of a compound having a mass of 175.0633 gram/mol, a compound having a mass of 208.0737 gram/mol, and a compound having a mass of 315.2409 gram/mol is determined in (a) and (b), the changes compared in (c) and analysed in (d).
44. The method according to any one of embodiments 25 to 30, wherein at least the amount of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol is determined in (a) and (b), the changes compared in (c) and analysed in (d)
45. The method according to any one of embodiments 25 to 30, wherein at least the amount of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol is determined in (a) and (b), the changes compared in (c) and analysed in (d).
46. A method for identifying a subject suffering from Alzheimer's dementia (AD) or being at risk of developing Alzheimer's dementia, the method comprising:
   (a) determining in a first body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
   (b) determining in a second body fluid sample of the subject the amount of the same at least one compound as in (a);
   (c) comparing the amount of the at least one compound determined according to (a) to the respective amount of the at least one compound determined according to (b), wherein a change of the amount of the at least one compound according to (b) compared with the amount as determined in (a) indicates Alzheimer's dementia for the subject or the risk of developing Alzheimer's dementia for the subject;
   wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.
47. The method for identifying a subject suffering from Alzheimer's dementia (AD) or being at risk of developing Alzheimer's dementia according to embodiment 46, wherein the at least one compound having a mass in gram/mol is selected from the group consisting of 110.0367, 232.004, 224.084, 254.1153, 189.9936, and 189.0096 (Group 2).
48. The method for identifying a subject suffering from Alzheimer's dementia (AD) or being at risk of developing Alzheimer's dementia according to embodiment 46 or 47, wherein in (a) and (b) at least the amount of a compound having a mass of 110.0367 gram/mol is determined and compared in (c).
49. The method for identifying a subject suffering from Alzheimer's dementia (AD) or being at risk of developing Alzheimer's dementia according to any one of embodiments 46 to 48, wherein the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in steps (a) and (b) and compared in (c), wherein a change of the amount of each of the at least two compounds, preferably each of the at least three compounds, more preferably each of the at least four compounds, more preferably each of the at least five compounds, according to (b) compared with the amount as determined in (a) indicates Alzheimer's dementia for the subject or the risk of developing Alzheimer's dementia for the subject, wherein more preferably one of the at least two compounds, preferably one of the at least three compounds, more preferably one of the at least four compounds, more preferably one of the at least five compounds is the compound having a mass of 110.0367 gram/mol.
50. The method for identifying a subject suffering from Alzheimer's dementia (AD) or being at risk of developing Alzheimer's dementia according to any one of embodiments 46 to 49, wherein the amount of at least two compounds, preferably at least six compounds or at least seven compounds, having a mass in gram/mol selected from the group consisting of Group 1 or Group 2 is determined in a first body fluid sample of the subject in steps (a) and (b) and compared in (c), wherein a change of the amount of each of the at least two compounds, preferably each of the at least six compounds, more preferably each of the at least seven compounds, according to (b) compared with the amount as determined in (a) indicates Alzheimer's dementia for the subject or the risk of developing Alzheimer's dementia for the subject, wherein more preferably one of the at least two compounds, more preferably one of the at least six compounds or one of the at least seven compounds, is the compound having a mass of 110.0367 gram/mol.
51. The method for identifying a subject suffering from Alzheimer's dementia (AD) or being at risk of developing Alzheimer's dementia according to any one of embodiments 46 to 50, wherein the amount of at least one further compound having a mass in gram/mol selected from the group consisting of 208.0737, 315.2409, 175.0633, 702.5682, 161.0477, and 97.9769 (Group 3), more preferably from the group consisting of 208.0737, 315.2409, 175.0633, 702.5682 (Group 4), is additionally determined in a first body fluid sample of the subject in steps (a) and (b) and compared in (c), wherein a change of the amount of the at least one compound according to (b) compared with the amount as determined in (a) and a change of the amount of the at least one further compound according to (b) compared with the amount as determined in (a) indicates Alzheimer's dementia for the subject or the risk of developing Alzheimer's dementia for the subject.
52. A method for identifying a subject suffering from Alzheimer's dementia (AD) or being at risk of developing Alzheimer's dementia, comprising
   (a) determining in a first body fluid sample of the subject the amount of at least two compounds having a mass in gram/mol selected from the group consisting of Group 3;
   (b) determining in a second body fluid sample of the subject the amount of the same at least two compound as in (a);
   (c) comparing the amounts of the at least two compounds determined according to (a) to the respective amounts of the at least two compounds determined according to (b), wherein a change of the amounts of the at least two compounds according to (b) compared with the amount as determined in (a) indicates Alzheimer's dementia for the subject or the risk of developing Alzheimer's dementia for the subject;
   wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.
53. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 189.0096 gram/mol, a compound having a mass of 224.084 gram/mol, and a compound having mass of 254.1153 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
54. The method for identifying a subject suffering from Alzheimer's dementia (AD) or being at risk of developing Alzheimer's dementia according to any one of embodiments 46 to 51, wherein at least the amounts of a compound having a mass of 189.993 gram/mol, a compound having a mass of 224.084 gram/mol, a compound having a mass of 232.004 gram/mol and a compound having a mass of 254.115 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
55. The method for identifying a subject suffering from Alzheimer's dementia (AD) or being at risk of developing Alzheimer's dementia according to any one of embodiments 46 to 51, wherein at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
56. The method for identifying a subject suffering from Alzheimer's dementia (AD) or being at risk of developing Alzheimer's dementia according to any one of embodiments 46 to 51, wherein at least the amounts of a compound having a mass of 175.0633 gram/mol, a compound having a mass of 208.0737 gram/mol and a compound having a mass of 254.1153 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.
57. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
58. The method for identifying a subject suffering from Alzheimer's dementia (AD) or being at risk of developing Alzheimer's dementia according to any one of embodiments 46 to 51, wherein at least the amounts of a compound having a mass of 175.0633 gram/mol, a mass of 208.0737 gram/mol, a compound having a mass of 315.2409 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.
59. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 175.0633 gram/mol, and a compound having a mass of 315.2409 gram/mol, are determined in a first body fluid sample of the subject in step (a) and (b) and compared in (c), wherein a change of the amounts of each of these compounds according to (b) compared with the amounts as determined in (a) by at least 2% indicates Alzheimer's dementia for the subject or the risk of developing Alzheimer's dementia for the subject.
60. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
61. The method for identifying a subject suffering from Alzheimer's dementia (AD) or being at risk of developing Alzheimer's dementia according to any one of embodiments 46 to 51, wherein at least the amounts of a compound having a mass of 175.0633 gram/mol, and a compound having a mass of 208.0737 gram/mol are determined in a first body fluid sample of the subject in step (a) and (b) and compared in (c), wherein a change of the amounts of each of these compounds according to (b) compared with the amounts as determined in (a) by at least 12.5% indicates Alzheimer's dementia for the subject or the risk of developing Alzheimer's dementia for the subject.
62. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 175.0633 gram/mol, and a compound having a mass of 208.0737 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
63. The method for identifying a subject suffering from Alzheimer's dementia (AD) or being at risk of developing Alzheimer's dementia according to any one of embodiments 46 to 51, wherein at least the amounts of a compound having a mass of 208.0737 gram/mol, and a compound having a mass of 702.5682 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
64. The method for identifying a subject suffering from Alzheimer's dementia (AD) or being at risk of developing Alzheimer's dementia according to any one of embodiments 46 to 51, wherein at least the amounts of a compound having a mass of 175.0633 gram/mol, a compound having a mass of 208.0737 gram/mol, and a compound having a mass of 315.2409 gram/mol are determined in a first body fluid sample of the subject in step (a) and (b) and compared in (c), wherein a change of the amounts of each of these compounds according to (b) compared with the amounts as determined in (a) by at least 40% indicates Alzheimer's dementia for the subject or the risk of developing Alzheimer's dementia for the subject.
65. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in a first body fluid sample of the subject in step (a) and (b) and compared in (c), wherein a change of the amounts of each of these compounds according to (b) compared with the amounts as determined in (a) by at least 1% indicates Alzheimer's dementia for the subject or the risk of developing Alzheimer's dementia for the subject.
66. The method for identifying a subject suffering from Alzheimer's dementia (AD) or being at risk of developing Alzheimer's dementia according to any one of embodiments 46 to 51, wherein at least the amounts of a compound having a mass of 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in a first body fluid sample of the subject in step (a) and (b) and compared in (c), wherein a change of the amounts of each of these compounds according to (b) compared with the amounts as determined in (a) by at least 2% indicates Alzheimer's dementia for the subject or the risk of developing Alzheimer's dementia for the subject.
67. A method for identifying a subject suffering from Alzheimer's dementia (AD), the method comprising:
   (a) determining in a body fluid sample of the subject the amount of at least one compound having a mass in gram/mol selected from the group consisting of 110.0367, 232.004, 224.084, 254.1153, 189.9936, 189.0096, 155.007, 355.345, 358.159, 109.0706, 110.0004, 145.0226, 166.0633, 178.0995, 190.0631, 191.0253, 200.0452, 212.0536, 234.1832, 236.1049, 259.9982, 285.1362, 291.0832, 301.1313, 314.1266, 318.1215, 319.2486, 331.2545, 332.1372, 334.2146, 339.3135, 346.1528, 350.0956, 366.0912, 371.8758, 440.2751, 538.1752, 540.2563, and 782.5638 (Group 1);
   (b) comparing the amount of the at least one compound determined according to (a) to a mean amount of a healthy subject, wherein a change of the amount of the at least one compound according to (a) compared with the mean amount of a healthy subject, preferably by at least 3% based on the mean amount of a healthy subject being 100%, indicates Alzheimer's dementia for the subject or indicates that the subject is at risk of developing Alzheimer's dementia;
   wherein said body fluid sample had been obtained after the subject had been administered thiethylperazine or a pharmaceutically acceptable salt thereof.
68. The method for identifying a subject suffering from Alzheimer's dementia (AD) or being at risk of developing Alzheimer's dementia according to embodiment 67, wherein the least one compound having a mass in gram/mol is selected from the group consisting of 110.0367, 232.004, 224.084, 254.1153, 189.9936, and 189.0096 (Group 2).
69. The method for identifying a subject suffering from Alzheimer's dementia (AD) or being at risk of developing Alzheimer's dementia according to embodiment 67 or 68, wherein in (a) and (b) at least the amount of a compound having a mass in gram/mol of 110.0367 determined and compared in (c).
70. The method for identifying a subject suffering from Alzheimer's dementia (AD) or being at risk of developing Alzheimer's dementia according to any one of embodiments 67 to 69, wherein the amount of at least two compounds, preferably at least three compounds, more preferably at least four compounds, more preferably at least five compounds, having a mass in gram/mol selected from the group consisting of Group 1 or Group 2 is determined in a first body fluid sample of the subject in step (a) and compared in (b), wherein a change of the amount of each of the at least two compounds, preferably each of the at least three compounds, more preferably each of the at least four compounds, more preferably of the at least five compounds, indicates Alzheimer's dementia for the subject or the risk of developing Alzheimer's dementia for the subject, wherein more preferably one of the at least two compounds, preferably one of the at least three compounds, more preferably one of the at least four compounds, more preferably one of the at least five compounds is the compound having a mass in gram/mol of 110.0367.
71. The method for identifying a subject suffering from Alzheimer's dementia (AD) or being at risk of developing Alzheimer's dementia according to any one of embodiments 67 to 70, wherein the amount of at least two compounds, preferably at least six compounds, more preferably at least seven compounds, having a mass in gram/mol selected from the group consisting of Group 1 or Group 2 is determined in a first body fluid sample of the subject in step (a) and compared in (b), wherein a change of the amount of each of the at least two compounds, preferably each of the at least six compounds, more preferably each of the at least seven compounds, indicates Alzheimer's dementia for the subject or the risk of developing Alzheimer's dementia for the subject, wherein more preferably one of the at least two compounds, more preferably one of the at least six compounds or one of the at least seven compounds, is the compound having a mass in gram/mol of 110.0367 . . .
72. The method for identifying a subject suffering from Alzheimer's dementia (AD) or being at risk of developing Alzheimer's dementia according to any one of embodiments 67 to 71, wherein the amount of at least one further compound having a mass in gram/mol selected from the group consisting of 208.0737, 315.2409, 175.0633, 702.5682, 161.0477, and 97.9769 (Group 3), more preferably from the group consisting of 208.0737, 315.2409, 175.0633, 702.5682 (Group 4), is determined in a first body fluid sample of the subject in steps (a) and (b) and compared in (c), wherein a change of the amount of the at least one compound according to (b) compared with the amount as determined in (a) and a change of the amount of the at least one further compound according to (a) compared with the mean amount of a healthy subject, preferably by at least 3% based on the mean amount of a healthy subject being 100% indicates Alzheimer's dementia for the subject or the risk of developing Alzheimer's dementia for the subject.
73. A method for identifying a subject suffering from Alzheimer's dementia (AD) or being at risk of developing Alzheimer's dementia, the method comprising:
   (a) determining in a body fluid sample of the subject the amounts of at least two compounds having a mass in gram/mol selected from the group consisting of Group 3, preferably of Group 4;
   (b) comparing the amounts of the at least two compounds determined according to (a) to a mean amount of a healthy subject, wherein a change of the amounts of the at least two compounds according to (a) compared with the mean amount of a healthy subject, preferably by at least 3% based on the mean amount of a healthy subject being 100%, indicates Alzheimer's dementia for the subject or indicates that the subject is at risk of developing Alzheimer's dementia;
   wherein said body fluid sample had been obtained after the subject had been administered thiethylperazine or a pharmaceutically acceptable salt thereof.
74. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 189.0096 gram/mol, a compound having a mass of 224.084 gram/mol, and a compound having a mass of 254.1153 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
75. The method for identifying a subject suffering from Alzheimer's dementia (AD) or being at risk of developing Alzheimer's dementia according to any one of embodiments 67 to 72, wherein at least the amounts of a compound having a mass of 189.993 gram/mol, a compound having a mass of 224.084 gram/mol, a compound having a mass of 232.004 gram/mol and a compound having a mass of 254.115 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
76. The method for identifying a subject suffering from Alzheimer's dementia (AD) or being at risk of developing Alzheimer's dementia according to any one of embodiments 67 to 72, wherein at least the amounts of a compound having a mass of 175.0633 gram/mol, a compound having a mass of 224.084 gram/mol, a compound having a mass of 232.004 gram/mol, a compound having a mass of 315.2409 gram/mol, and a compound having a mass of 702.5682 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
77. The method for identifying a subject suffering from Alzheimer's dementia (AD) or being at risk of developing Alzheimer's dementia according to any one of embodiments 67 to 72, wherein at least the amounts of a compound having a mass of 175.0633 gram/mol, a compound having a mass of 208.0737 gram/mol and a compound having a mass of 254.1153 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
78. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 175.0633 gram/mol, a compound having a mass of 224.084 gram/mol, a compound having a mass of 254.1153 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
79. The method for identifying a subject suffering from Alzheimer's dementia (AD) or being at risk of developing Alzheimer's dementia according to any one of embodiments 67 to 72, wherein at least the amounts of a compound having a mass of 175.0633 gram/mol, a compound having a mass of 208.0737 gram/mol, a compound having a mass of 315.2409 gram/mol, and a compound having a mass of 702.5682 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
80. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 175.0633 gram/mol, and a compound having a mass of 315.2409 gram/mol, are determined in a first body fluid sample of the subject in step (a) and compared in (b), wherein a change of the amounts of each of these compound by at least 3% indicates Alzheimer's dementia for the subject or the risk of developing Alzheimer's dementia for the subject.
81. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 208.0737 gram/mol, a compound having a mass of 232.004 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
82. The method for identifying a subject suffering from Alzheimer's dementia (AD) or being at risk of developing Alzheimer's dementia according to any one of embodiments 67 to 72, wherein at least the amounts of a compound having a mass of 175.0633 gram/mol, and a compound having a mass of 208.0737 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
83. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 175.0633 gram/mol, and a compound having a mass of 208.0737 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
84. The method for identifying a subject suffering from Alzheimer's dementia (AD) or being at risk of developing Alzheimer's dementia according to any one of embodiments 67 to 72, wherein at least the amounts of a compound having a mass of 208.0737 gram/mol, and a compound having a mass of 702.5682 gram/mol are determined in a first body fluid sample of the subject in step (a) and compared in (b), wherein a change of the amounts of each of these compounds by at least 12% indicates Alzheimer's dementia for the subject or the risk of developing Alzheimer's dementia for the subject.
85. The method for identifying a subject suffering from Alzheimer's dementia (AD) or being at risk of developing Alzheimer's dementia according to any one of embodiments 67 to 72, wherein at least the amounts of a compound having a mass of 175.0633 gram/mol, a compound having a mass of 208.0737 gram/mol, and a compound having a mass of 315.2409 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
86. The method for identifying a subject suffering from Alzheimer's dementia (AD) or being at risk of developing Alzheimer's dementia according to any one of embodiments 67 to 72, wherein at least the amounts of a compound having a mass of 110.0367 gram/mol, a compound having a mass of 232.004 gram/mol, a compound having a mass of 254.1153 gram/mol, a compound having a mass of 189.9936 gram/mol, a compound having a mass of 189.0096 gram/mol, and a compound having a mass of 224.084 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
87. The method for identifying a subject suffering from Alzheimer's dementia (AD) or being at risk of developing Alzheimer's dementia according to any one of embodiments 67 to 72, wherein at least the amounts of a compound having a mass 110.0367 gram/mol, a compound having a mass of 110.0367 gram/mol, a compound having a mass of 175.0633 gram/mol, a compound having a mass of 702.5682 gram/mol, a compound having a mass of 208.0737 gram/mol, a compound having a mass of 315.2409 gram/mol, a compound having a mass of 232.004 gram/mol, and a compound having a mass of 254.1153 gram/mol are determined in a first body fluid sample of the subject in step (a) and compared in (b), wherein a change of the amounts of each of these compounds by at least 3% indicates Alzheimer's dementia for the subject or the risk of developing Alzheimer's dementia for the subject.
88. The method for identifying a subject suffering from Alzheimer's dementia (AD) or being at risk of developing Alzheimer's dementia according to any one of embodiments 67 to 72, wherein the mean amount of a healthy subject value is a reference value of a healthy subject from literature, or a reference value previously determined from healthy subjects or a reference value from a body fluid sample of the subject prior to TEP administration.
89. A method according to any one of embodiments 1 to 88, wherein the amount determination in any one of steps (a) and/or (b) is done by mass spectrometry (MS), preferably by a tandem mass spectrometry (MS/MS), more preferably by a tandem mass spectrometry in combination with a preceding liquid chromatography (LC-MS/MS).
90. The method according to any one of embodiments 1 to 89, which is an in vitro method.
91. The method of any one of embodiments 1 to 90, wherein the subject is human.
92. The method of any one of embodiments 1 to 91, wherein the body fluid sample is selected from the group consisting of blood sample, urine sample, liquor sample (CSF) and mixtures of two or more thereof, preferably the body fluid sample is a blood sample; and respectively the first body fluid sample and the second body fluid sample are independently of each other selected from the group consisting of blood sample, urine sample, liquor sample (CSF) and mixtures of two or more thereof, wherein preferably, the first body fluid sample and the second body fluid sample are the same kind of sample and are both selected from the group consisting of blood sample, urine sample, liquor sample (CSF) and mixtures of two or more thereof, wherein more preferably, first body fluid sample and second body fluid sample are both each a blood sample (first blood sample and second blood sample).
93. At least one compound having a mass in gram/mol selected from the group consisting of Group 1, or (Group 2), or as defined in any one of embodiments 1 to 92 and optionally in combination with at least one compound having a mass in gram/mol selected from the group consisting of Group 3, preferably of Group 4, for diagnosing AD in a subject or for determining the risk of a subject to develop AD.
94. The at least one compound for use according to embodiment 93 wherein at least two compounds having a mass in gram/mol selected from the group consisting of Group 1, or
   Group 2 as defined in any one of embodiments 1 to 92, preferably at least three compounds having a mass in gram/mol selected from the group consisting of Group 1, or
   Group 2 as defined in any one of embodiments 1 to 92, more preferably at least four compounds having a mass in gram/mol selected from the group consisting of Group 1, or
   Group 2 as defined in any one of embodiments 1 to 92; more preferably at least five compounds having a mass in gram/mol selected from the group consisting of Group 1, or
   Group 2 as defined in any one of embodiments 1 to 92;
   each optionally in combination with at least one compound having a mass in gram/mol selected from the group consisting of Group 3 or Group 4, are used.
95. The at least one compound for use according to embodiment 93, wherein at least two compounds having a mass in gram/mol selected from the group consisting of Group 1, or
   Group 2 as defined in any one of embodiments 1 to 92, preferably at least six compounds having a mass in gram/mol selected from the group consisting of Group 1, or Group 2 as defined in any one of embodiments 1 to 92, more preferably at least seven compounds having a mass in gram/mol selected from the group consisting of Group 1, or Group 2 as defined in any one of embodiments 1 to 92;
   each optionally in combination with at least one compound having a mass in gram/mol selected from the group consisting of Group 3 or Group 4, are used.
96. At least two compounds having a mass in gram/mol selected from the group consisting of Group 3 or Group 4 for diagnosing AD in a subject or for determining the risk of a subject to develop AD.

### 2^{nd} Diagnostic Use (Alternative Wording)

97. Use of at least one compound having a mass in gram/mol selected from the group consisting of Group 1, or Group 2 as defined in any one of embodiments 1 to 92 and optionally in combination with at least one compound having a mass in gram/mol selected from the group consisting of Group 3 or Group 4 in a sample of a subject for diagnosing AD in said subject or for determining the risk of said subject to develop AD.
98. The use according to embodiment 97, wherein at least two, preferably at least three, more preferably at least four, more preferably at least five, compounds having a mass in gram/mol selected from the group consisting of Group 1, or Group 2 as defined in any one of embodiments 1 to 93 and optionally in combination with at least one compound having a mass in gram/mol selected from the group consisting of Group 3 or Group 4 in a sample of a subject are used for diagnosing AD in said subject or for determining the risk of said subject to develop AD.
99. The use according to embodiment 97, wherein at least two, preferably at least six, more preferably at least seven, compounds having a mass in gram/mol selected from the group consisting of Group 1, or Group 2 as defined in any one of embodiments 1 to 92 and optionally in combination with at least one compound having a mass in gram/mol selected from the group consisting of Group 3 or Group 4 in a sample of a subject are used for diagnosing AD in said subject or for determining the risk of said subject to develop AD.
100. Use of at least two compounds having a mass in gram/mol selected from the group consisting of Group 3 or group 4 in a sample of a subject for diagnosing AD in said subject or for determining the risk of said subject to develop AD.
101. A device adapted for identifying a subject suffering from Alzheimer's dementia (AD) or being at risk of developing Alzheimer's dementia, the device comprising:
   (a) an analyzing unit capable of determining in a first body fluid sample of the subject the amount of at least one compound having a mass in gram/mol selected from the group consisting of Group 1;
   (b) a data processor adopted for analyzing the determined amount for the at least one compound using a prediction algorithm implemented in said data processor that is capable of predicting AD in a subject based on the at least two compounds, whereby a subject suffering from AD is identified if AD is predicted or a subject being at risk of developing Alzheimer's dementia is identified if AD is predicted.
102. A kit for identifying a subject suffering from Alzheimer's dementia (AD) or being at risk of developing Alzheimer's dementia, the kit comprising a reference sample of at least one compound having a mass in gram/mol selected from the group consisting of Group 1 or Group 2, optionally a reference sample of at least one compound having a mass in gram/mol selected from the group consisting of Group 3, or optionally a reference sample of at least one compound having a mass in gram/mol selected from the group consisting of Group 4, and optionally one or more reagents for sample preparation and/or extraction.

The present invention is further illustrated by the following reference examples, comparative examples, and examples.

### EXAMPLES

The following Examples shall merely illustrate the invention. Whatsoever, they shall not be construed as limiting the scope of the invention.

### I Methods

### I.1 Treatment

15 human subjects (7 healthy, 8 suffering from Alzheimer's disease) were treated with 6.5 mg of thiethylperazine (TEP). Blood samples were taken 0, 0.5, 1, 1.5, 2, 4 and 8 hours after administration. AD subjects were classified by a Mini-Mental State Examination (MMSE) Score of 25-18. AD diagnosis was furthermore confirmed by recommended examinations per German DGN/DGPPN S3 Guideline “Dementia” and by:
psychometric and cognitive tests
lumbar puncture in subjects with uncertain AD diagnosis following CNS imaging
Clinical Dementia Rating (global CDR) of 0.5 or 1 and memory box score of at least 0.5.
Isolated or predominant episodic memory deficit, manifested as memory performance in the Logical Memory subscale (Delayed Paragraph Recall) from the Wechsler Memory Scale-III of 1 standard deviation below age-adjusted norms.

### I.2 Blood Sampling

Samples of 10 ml venous blood were collected in standardized test tubes containing EDTA and gently shaken 1-2 times. As soon as possible (within max. 30 min.) samples were centrifuged at 1600 g, room temperature for 10 min. Plasma was aliquoted into 6 portions of 0.5 ml. All aliquots were frozen into a deep freezer (−80 °C) within 10 min after the centrifugation and stored until use.

### I.3 Untargeted Metabolomics

All procedures done in this section were performed by BGI Genomics. Untargeted metabolomics refers to the high-throughput analysis of the metabolic state of a biological system (see Matthaios-Emmanouil P Papadimitropoulos Catherine G Vasilopoulou Christoniki Maga-Nteve, Maria I Klapa in Methods Mol Biol. 2018; 1738:133-147; doi: 10.1007/978-1-4939-7643-0_9.).

### 1 Main Instruments and Reagents

Ultra Performance Liquid Chromatography: Waters 2D UPLC, Waters, USA
High Resolution mass spectrometer: Q Exactive, Thermo Fisher Scientific, USA
Chromatographic Column: ACQUITY UPLC BEH C18 1.7 μm, 2.1*100 mm, Waters, USA
Low Temperature High Speed Centrifuge: Centrifuge 5430, Eppendorf
Vortex: QL-901, Kylin-bell Lab Instruments Co., Ltd., China
Ultrapure Water Systems: Milli-Q Integral, Millipore Corporation, USA
Vacuum Concentrator: Maxi Vacbeta, GENE COMPANY
The internal standard mix (IS) contains: L-Leucine-d3, L-Phenylalanine (13C9, 99%),
LTryptophan-d5, Progesterone-2,3,4-13C3
MS-grade methanol (A454-4) and acetonitrile (A996-4) were purchased from Thermo Fisher Scientific (USA). Formic acid was purchased from DIMKA (50144-50 ml, USA) and ammonium formate (17843-250G, Honeywell Fluka, USA) was obtained from Fluka. Ultrapure water was filtered through the Milli-Q system.

### 2 Metabolite Extraction

Metabolite extraction was primarily performed according to previously reported methods [4] [5]. In short, 100 μL plasma samples were extracted by directly adding 300 μL of precooled methanol and acetonitrile (2:1, v/v), internal standards mix 1 (IS1) and internal standards mix 2 (IS2) were added for quality control of sample preparation. After Vortex for 1 min and incubate at −20 °C for 2 hours, samples were centrifuged for 20 min at 4000 rpm, and the supernatant was then transferred for vacuum freeze drying. The metabolites were resuspended in 150 μL of 50% methanol and centrifuged for 30 min at 4000 rpm, and the supernatants were transferred to autosampler vials for LC-MS analysis. A quality control (QC) sample was prepared by pooling the same volume of each sample to evaluate the reproducibility of the whole LC-MS analysis.

### 3 LC-MS/MS Analysis

### 3.1. Chromatographic Conditions

The samples were analyzed on a Waters 2D UPLC (Waters, USA), coupled to a Q-Exactive mass spectrometer (Thermo Fisher Scientific, USA) with a heated electrospray ionization (HESI) source and controlled by the Xcalibur 2.3 software program (Thermo Fisher Scientific, Waltham, MA, USA). Chromatographic separation was performed on a Waters ACQUITY UPLC BEH C18 column (1.7 μm, 2.1 mm×100 mm, Waters, USA), and the column temperature was maintained at 45 °C. The mobile phase consisted of 0.1% formic acid (A) and acetonitrile (B) in the positive mode, and in the negative mode, the mobile phase consisted of 10 mM ammonium formate (A) and acetonitrile (B). The gradient conditions were as follows: 0-1 min, 2% B; 1-9 min, 2%-98% B; 9-12 min, 98% B; 12-12.1 min, 98% B to 2% B; and 12.1-15 min, 2% B. The flow rate was 0.35 mL/min and the injection volume was 5 μL.

### 3.2 Mass Spectrometry Conditions

The mass spectrometric settings for positive/negative ionization modes were as follows: spray Voltage: 3.8/−3.2 kV; sheath gas flow rate: 40 arbitrary units (arb); aux gas flow rate: 10 arb; aux gas heater temperature: 350 °C; capillary temperature: 320 °C. The full scan range was 70-1050 m/z with a resolution of 70,000, and the automatic gain control (AGC) target for MS acquisitions was set to 3e6 with a maximum ion injection time of 100 ms. Top 3 precursors were selected for subsequent MSMS fragmentation with a maximum ion injection time of 50 ms and resolution of 17,500, the AGC was 1e5. The stepped normalized collision energy was set to 20, 40 and 60 eV.

In order to provide more reliable experimental results during instrument testing, the samples were randomly ordered to reduce system errors. A QC sample was interspersed for every 10 samples.

### 4 Data Preprocessing

The mass spectrometry raw data (raw file) collected by LC-MS/MS was imported into Compound Discoverer 3.1 (Thermo Fisher Scientific, USA) for data processing, including: peak extraction, retention time correction within and between groups, additive ion pooling, missing value filling, background peak labeling, and metabolite identification. Finally, information on compound molecular weight, retention time, peak area, and identification results were exported. The identification of metabolites is a combined result of BGI self-built standard library, mzCloud and ChemSpider (HMDB, KEGG, LipidMaps) databases.

The results of the Compound Discoverer 3.1 export are imported into metaX for data preprocessing, including: 1. Normalizing the data using the Probabilistic Quotient Normalization (PQN [6]) to obtain the relative peak area. 2. Correcting the batch effect using QC-RLSC [4] (Quality control-based robust LOESS signal correction); 3. Calculating the CV (Coefficient of Variation) of the relative peak area in all QC samples, and deleting the compounds with CV greater than 30%. Probabilistic Quotient Normalization (PQN): Probabilistic quotient normalization is the calculation of the average of all QC samples for each feature to obtain a reference vector; calculating the median between the reference vector and each sample and obtaining the coefficient vector associated with each sample, then normalization was performed by dividing each sample by the median of the coefficient vectors (the median of each sample is different).

QC-RLSC [4]: Local polynomial regression fitting signal correction (QC-RLSC) based on QC sample information.

Data quality was assessed by repeatability of QC sample detection. The content includes chromatogram overlap of the QC sample, PCA, peak number and peak response intensity difference.

### 5 Partial Least Squares-Discriminant Analysis (PLS-DA)

The partial least squares regression method was used to establish a relationship model between metabolite expression and sample categories to achieve modelling prediction of sample categories. The PLS-DA model between the comparative groups (two groups) was established after log 2-log conversion of the data, and the method used for scaling was Par. The 7-fold cross-validation was performed during modeling. To evaluate the model, the PLS-DA model [7] [8] was subjected to 200 response permutations.

Data were used for fold change analysis and T-test. Fold change (FC) was obtained through fold change analysis, p-value was obtained through Students T-test with p-values corrected for false discovery rate (FDR) to obtain q-value.

Comparison was done between healthy probands before administration of thiethylperazine versus probands having Alzheimer's disease before administration of thiethylperazine and healthy probands after administration of thiethylperazine versus probands having Alzheimer's disease after administration of thiethylperazine.

### 6 Databases and Identification

Databases used for compound identification included: mzCloud, Chemspider, Human Metabolome Database (HMDB), Kyoto Encyclopedia of Genes and Genomes (KEGG),

Lipidmaps database.

II Identification of Metabolites/Masses with Diagnostic Value

### 1. Software

Python 3.9.5 running in the Spyder 5.3.2 IDE with libraries: scikit-learn, pandas and numpy. Windows 11, Microsoft Excel 365.

### 2. Processing and Filtering of Data

After processing all blood samples using the untargeted metabolomics procedure, the area under the curve (AUC) value of every mass (x) was standardized using the formula

z=(x−u)/s
where u was the mean of all samples and s was the standard deviation of all samples. Standardized data represent dataset 1.

Afterwards, a correlation analyses of all masses within dataset 1 was performed. In case an absolute pairwise correlation of more than 0.95 was found, one mass of the pair was excluded from further analysis. The remaining data represent dataset 2.

Dataset 2 was then consecutively filtered for (i) masses that are not significantly different between healthy and AD subjects at the pre-TEP time point and (ii) masses that are significantly different at the post-TEP time point according to Students T-test after running PLS-DA to produce dataset 3.

Dataset 3 was then checked for masses that were found to have highly correlating masses in dataset 1. Four such masses were identified and the correlating masses, excluded from dataset 2, were reintroduced into dataset 3 to give the final dataset comprising 45 masses.

### 3. Analyzing Predictive Value of Single Masses

For better visualization, AUC data at 2 h after TEP administration of each of the selected 45 masses was normalized to the respective mean of the healthy individuals (X_{mass i}=AUC_{mass i}*100/Average_{all Healthy mass i}). 32 masses were significantly different in samples acquired from AD subjects than in healthy subjects' samples. For 42 of the 45 masses, an absolute change of 3% (relative to the mean of healthy subjects) was sufficient to diagnose AD with an accuracy of at least 67% (Table 1).

TABLE 1

| Mass | | | | | |
|---|---|---|---|---|---|
| [g/mol] | 97.9769 | 109.0706 | 110.0004 | 110.0367 | 145.0226 |
| AD [%] | 70.7889397 | 100.29137 | 116.828082 | 58.547488 | 115.089498 |
| AD [%] | 67.6508564 | 106.293315 | 159.644731 | 64.3186048 | 105.084437 |
| AD [%] | 107.88692 | 105.896656 | 133.762571 | 90.6379443 | 115.731424 |
| AD [%] | 60.6132804 | 127.042729 | 85.1334398 | 97.0477313 | 99.1105245 |
| AD [%] | 48.7746399 | 138.373199 | 134.510593 | 58.5715032 | 115.403101 |
| AD [%] | 73.1471474 | 141.80394 | 120.450171 | 65.4292133 | 108.204659 |
| AD [%] | 91.9834229 | 130.105098 | 123.478532 | 98.1299587 | 108.165366 |
| AD [%] | 47.7778671 | 134.112894 | 118.432278 | 76.057998 | 107.806084 |
| Healthy | 79.4725056 | 93.9118234 | 119.890036 | 80.8695173 | 101.547743 |
| [%] | | | | | |
| Healthy | 114.927384 | 90.1013512 | 94.2603139 | 107.061303 | 95.9504528 |
| [%] | | | | | |
| Healthy | 106.229045 | 89.1614703 | 86.008959 | 86.6353542 | 95.2608344 |
| [%] | | | | | |
| Healthy | 106.074135 | 91.973342 | 123.671858 | 97.1926102 | 92.0673347 |
| [%] | | | | | |
| Healthy | 110.187263 | 140.878126 | 111.72562 | 145.406611 | 101.896489 |
| [%] | | | | | |
| Healthy | 112.013099 | 99.1872102 | 86.6392924 | 92.6249548 | 95.7175644 |
| [%] | | | | | |
| Healthy | 71.0965679 | 94.7866768 | 77.8039205 | 90.2096491 | 117.559581 |
| [%] | | | | | |
| Mean Healthy | 100.0 | 100.0 | 100.0 | 100.0 | 100.0 |
| [%] | | | | | |
| SEM Healthy | 6.6 | 6.9 | 6.9 | 8.2 | 3.2 |
| [%] | | | | | |
| Mean AD | 71.1 | 123.0 | 124.0 | 76.1 | 109.3 |
| [%] | | | | | |
| SEM AD | 7.3 | 5.8 | 7.4 | 6.0 | 2.1 |
| [%] | | | | | |
| p-value | 0.0119 | 0.0233 | 0.0352 | 0.0321 | 0.0264 |
| Cut-Off | <97 | >103 | >103 | <92 | >103 |
| [%] | | | | | |
| Accuracy | 80 | 87 | 73 | 67 | 87 |
| smallest | 7.89 | 0.29 | 14.87 | 1.87 | 0.89 |
| absolute | | | | | |
| change AD | | | | | |
| [%] | | | | | |

| Mass | | | | | |
|---|---|---|---|---|---|
| [g/mol] | 155.007 | 161.0477 | 166.0633 | 175.0633 | 178.0995 |
| AD [%] | 125.175311 | 34.3452217 | 115.910192 | 141.172482 | 31.6133904 |
| AD [%] | 105.533431 | 76.9755165 | 102.454969 | 127.440386 | 45.8051346 |
| AD [%] | 107.171274 | 52.1919166 | 111.69906 | 145.569006 | 57.0090766 |
| AD [%] | 99.1370008 | 54.879389 | 220.171043 | 141.835109 | 70.3564955 |
| AD [%] | 114.127976 | 55.2166038 | 368.871772 | 100.527094 | 20.1104847 |
| AD [%] | 110.776006 | 62.4615942 | 139.40362 | 123.480836 | 60.390134 |
| AD [%] | 119.36387 | 27.168683 | 263.803736 | 117.897587 | 116.86444 |
| AD [%] | 113.542187 | 52.9990746 | 122.923942 | 136.250751 | 22.9513469 |
| Healthy | 106.470641 | 49.7390484 | 119.965224 | 113.52465 | 87.2724877 |
| [%] | | | | | |
| Healthy | 91.1971838 | 37.2177991 | 198.833265 | 87.1876702 | 76.674093 |
| [%] | | | | | |
| Healthy | 101.804551 | 110.269281 | 75.1940137 | 107.724756 | 119.631851 |
| [%] | | | | | |
| Healthy | 91.2787198 | 110.685275 | 102.635891 | 99.3438269 | 110.414407 |
| [%] | | | | | |
| Healthy | 96.8973056 | 199.160244 | 113.109689 | 92.9531641 | 144.871841 |
| [%] | | | | | |
| Healthy | 105.225655 | 69.8282377 | 46.45414 | 79.0680513 | 76.6835489 |
| [%] | | | | | |
| Healthy | 107.125944 | 123.100115 | 43.8077775 | 120.197882 | 84.4517721 |
| [%] | | | | | |
| Mean Healthy | 100.0 | 100.0 | 100.0 | 100.0 | 100.0 |
| [%] | | | | | |
| SEM Healthy | 2.6 | 20.7 | 20.1 | 5.6 | 9.8 |
| [%] | | | | | |
| Mean AD | 111.9 | 52.0 | 180.7 | 129.3 | 53.1 |
| [%] | | | | | |
| SEM AD | 2.9 | 5.5 | 33.8 | 5.4 | 11.1 |
| [%] | | | | | |
| p-value | 0.0101 | 0.0330 | 0.0700 | 0.0023 | 0.0081 |
| Cut-Off | >103 | <97 | >103 | >103 | <97 |
| [%] | | | | | |
| Accuracy | 73 | 80 | 73 | 73 | 67 |
| smallest | 0.86 | 23.02 | 2.45 | 0.53 | 16.86 |
| absolute | | | | | |
| change AD | | | | | |
| [%] | | | | | |

| Mass | | | | | | |
|---|---|---|---|---|---|---|
| [g/mol] | 189.0096 | 189.9936 | 190.0631 | 191.0253 | 200.0452 | 208.0737 |
| AD [%] | 82.3015605 | 59.4991544 | 41.9891965 | 386.21709 | 130.318799 | 42.525242 |
| AD [%] | 4.74266801 | 62.0557515 | 4.62445349 | 140.915157 | 207.863605 | 4.09558136 |
| AD [%] | 63.745828 | 92.6717104 | 77.516061 | 166.34949 | 147.839717 | 79.7076005 |
| AD [%] | 43.0626521 | 101.619911 | 63.8585477 | 87.3152359 | 171.292756 | 67.0892009 |
| AD [%] | 34.3455656 | 61.8607668 | 31.1964736 | 172.167932 | 91.3092416 | 31.131329 |
| AD [%] | 66.8708051 | 67.330976 | 44.504938 | 157.393069 | 191.138732 | 46.93251 |
| AD [%] | 54.2651059 | 78.6072505 | 26.481317 | 188.606018 | 104.962436 | 26.2252034 |
| AD [%] | 82.4433515 | 77.9055429 | 47.5459402 | 135.888302 | 119.822399 | 49.8379264 |
| Healthy | 128.878441 | 83.3693259 | 85.6920173 | 197.422215 | 123.852096 | 87.2763896 |
| [%] | | | | | | |
| Healthy | 62.0547614 | 84.7532669 | 75.2703768 | 104.568223 | 76.7811184 | 71.0716595 |
| [%] | | | | | | |
| Healthy | 87.0484121 | 82.052037 | 45.1268753 | 87.25741 | 85.1761072 | 44.9758594 |
| [%] | | | | | | |
| Healthy | 64.2765814 | 121.868578 | 136.588334 | 79.7072146 | 87.5829429 | 133.362822 |
| [%] | | | | | | |
| Healthy | 179.254813 | 141.094406 | 188.194368 | 45.4993365 | 49.6902918 | 192.408747 |
| [%] | | | | | | |
| Healthy | 97.5479353 | 97.3001992 | 69.8340128 | 44.5717026 | 107.891641 | 67.2139745 |
| [%] | | | | | | |
| Healthy | 80.9390559 | 89.5621866 | 99.2940159 | 140.973898 | 169.025803 | 103.690548 |
| [%] | | | | | | |
| Mean Healthy | 100.0 | 100.0 | 100.0 | 100.0 | 100.0 | 100.0 |
| [%] | | | | | | |
| SEM Healthy | 15.7 | 8.6 | 18.2 | 20.6 | 14.5 | 18.8 |
| [%] | | | | | | |
| Mean AD | 54.0 | 75.2 | 42.2 | 179.4 | 145.6 | 43.4 |
| [%] | | | | | | |
| SEM AD | 9.3 | 5.5 | 7.9 | 31.5 | 14.7 | 8.4 |
| [%] | | | | | | |
| p-value | 0.0219 | 0.0268 | 0.0092 | 0.0619 | 0.0471 | 0.0128 |
| Cut-Off | <97 | <97 | <97 | >103 | >103 | <97 |
| [%] | | | | | | |
| Accuracy | 73 | 67 | 73 | 73 | 73 | 73 |
| smallest | 17.56 | 1.62 | 12.48 | 12.68 | 8.69 | 20.29 |
| absolute | | | | | | |
| change AD | | | | | | |
| [%] | | | | | | |

| Mass | | | | |
|---|---|---|---|---|
| [g/mol] | 212.0536 | 224.084 | 232.004 | 234.1832 |
| AD [%] | 90.0227442 | 177.60883 | 84.0073191 | 110.097633 |
| AD [%] | 112.926753 | 108.946879 | 8.12468793 | 13.8684348 |
| AD [%] | 132.169153 | 112.448332 | 64.9862532 | 24.6555669 |
| AD [%] | 118.678287 | 103.751621 | 51.097393 | 1.17570689 |
| AD [%] | 123.411245 | 161.886465 | 31.0794113 | 4.38780301 |
| AD [%] | 147.139224 | 99.959267 | 61.4024287 | 20.5976208 |
| AD [%] | 137.753289 | 133.520259 | 48.3894475 | 21.1958883 |
| AD [%] | 114.957584 | 124.969551 | 74.3553642 | 22.3450429 |
| Healthy | 95.132348 | 137.859377 | 116.172396 | 58.0618318 |
| [%] | | | | |
| Healthy | 91.5717353 | 94.0305703 | 71.6371127 | 356.284653 |
| [%] | | | | |
| Healthy | 102.074313 | 80.2436066 | 77.0377535 | 35.2808634 |
| [%] | | | | |
| Healthy | 95.4149106 | 82.2574534 | 79.7312617 | 68.2744681 |
| [%] | | | | |
| Healthy | 92.794832 | 86.7453299 | 165.947268 | 24.6958378 |
| [%] | | | | |
| Healthy | 83.2707397 | 92.5341979 | 98.316056 | 135.480458 |
| [%] | | | | |
| Healthy | 139.741122 | 126.329465 | 91.1581521 | 21.9218879 |
| [%] | | | | |
| Mean Healthy | 100.0 | 100.0 | 100.0 | 100.0 |
| [%] | | | | |
| SEM Healthy | 7.0 | 8.6 | 12.4 | 45.2 |
| [%] | | | | |
| Mean AD | 122.1 | 127.9 | 52.9 | 27.3 |
| [%] | | | | |
| SEM AD | 6.2 | 10.0 | 8.6 | 12.2 |
| [%] | | | | |
| p-value | 0.0330 | 0.0579 | 0.0071 | 0.1225 |
| Cut-Off | >103 | >103 | <97 | <68 |
| [%] | | | | |
| Accuracy | 87 | 80 | 73 | 67 |
| smallest | 9.98 | 0.04 | 15.99 | 10.10 |
| absolute | | | | |
| change AD | | | | |
| [%] | | | | |

| Mass | | | | | |
|---|---|---|---|---|---|
| [g/mol] | 236.1049 | 254.1153 | 259.9982 | 285.1362 | 291.0832 |
| AD [%] | 159.520614 | 166.589488 | 140.774853 | 19.6997184 | 143.128398 |
| AD [%] | 67.6815549 | 74.8569285 | 108.571691 | 1.51937825 | 125.404568 |
| AD [%] | 112.168741 | 129.534518 | 137.744119 | 53.4757948 | 143.959916 |
| AD [%] | 209.994913 | 220.952093 | 117.281105 | 167.381175 | 111.566137 |
| AD [%] | 232.74869 | 247.193218 | 127.510866 | 6.44824518 | 151.283376 |
| AD [%] | 174.274482 | 183.456627 | 141.807789 | 22.3604874 | 165.485626 |
| AD [%] | 110.163639 | 125.464889 | 117.390529 | 24.7958567 | 84.5552299 |
| AD [%] | 111.503317 | 132.109119 | 129.007525 | 12.2492806 | 120.421947 |
| Healthy | 73.8247959 | 57.9673992 | 104.339893 | 46.3918122 | 90.5805719 |
| [%] | | | | | |
| Healthy | 197.289594 | 197.055248 | 94.9399234 | 143.476113 | 77.7604806 |
| [%] | | | | | |
| Healthy | 52.6097928 | 50.4868993 | 92.7531236 | 128.743702 | 97.2631692 |
| [%] | | | | | |
| Healthy | 110.599113 | 110.72796 | 90.4309506 | 74.2900115 | 108.151793 |
| [%] | | | | | |
| Healthy | 123.907715 | 131.385859 | 110.684071 | 180.705373 | 126.017495 |
| [%] | | | | | |
| Healthy | 91.4137916 | 93.2283163 | 90.326439 | 109.243865 | 102.465604 |
| [%] | | | | | |
| Healthy | 50.3551973 | 59.1483189 | 116.525599 | 17.1491228 | 97.7608863 |
| [%] | | | | | |
| Mean Healthy | 100.0 | 100.0 | 100.0 | 100.0 | 100.0 |
| [%] | | | | | |
| SEM Healthy | 19.3 | 19.8 | 4.0 | 21.7 | 5.7 |
| [%] | | | | | |
| Mean AD | 147.3 | 160.0 | 127.5 | 38.5 | 130.7 |
| [%] | | | | | |
| SEM AD | 20.0 | 19.8 | 4.3 | 19.2 | 9.1 |
| [%] | | | | | |
| p-value | 0.1149 | 0.0528 | 0.0005 | 0.0528 | 0.0157 |
| Cut-Off | >103 | >103 | >103 | <97 | >103 |
| [%] | | | | | |
| Accuracy | 73 | 73 | 80 | 73 | 80 |
| smallest | 10.16 | 25.14 | 8.57 | 46.52 | 11.57 |
| absolute | | | | | |
| change AD | | | | | |
| [%] | | | | | |

| Mass | | | | | |
|---|---|---|---|---|---|
| [g/mol] | 301.1313 | 314.1266 | 315.2409 | 318.1215 | 319.2486 |
| AD [%] | 2.32422864 | 40.4039422 | 188.250425 | 83.2291932 | 131.348623 |
| AD [%] | 0.40895734 | 70.5571797 | 254.76122 | 59.0292866 | 92.9571536 |
| AD [%] | 30.1730708 | 73.2726634 | 143.384456 | 32.8624498 | 172.234548 |
| AD [%] | 170.50454 | 50.795265 | 146.438766 | 43.0932799 | 90.3839111 |
| AD [%] | 0.43437768 | 85.7774209 | 137.580472 | 140.33125 | 156.466431 |
| AD [%] | 4.04144581 | 72.5644183 | 153.372733 | 78.2101859 | 138.996605 |
| AD [%] | 6.18768236 | 40.0183386 | 85.8899917 | 47.5360402 | 108.673606 |
| AD [%] | 1.2483797 | 57.7438227 | 220.831741 | 38.6602132 | 188.967355 |
| Healthy | 15.5687817 | 81.5782808 | 185.172223 | 153.619932 | 83.5627455 |
| [%] | | | | | |
| Healthy | 136.775644 | 56.8490831 | 114.512428 | 47.3788758 | 76.7003137 |
| [%] | | | | | |
| Healthy | 155.533893 | 92.4563691 | 114.155291 | 83.7487312 | 78.892998 |
| [%] | | | | | |
| Healthy | 55.6742716 | 104.387677 | 82.7688063 | 108.612339 | 111.55405 |
| [%] | | | | | |
| Healthy | 252.771792 | 174.238554 | 73.3293135 | 123.655191 | 85.39068 |
| [%] | | | | | |
| Healthy | 78.838845 | 98.576532 | 65.9690493 | 87.7887766 | 124.628075 |
| [%] | | | | | |
| Healthy | 4.83677331 | 91.9135043 | 64.0928886 | 95.196154 | 139.271138 |
| [%] | | | | | |
| Mean Healthy | 100.0 | 100.0 | 100.0 | 100.0 | 100.0 |
| [%] | | | | | |
| SEM Healthy | 33.2 | 13.7 | 16.3 | 12.6 | 9.5 |
| [%] | | | | | |
| Mean AD | 26.9 | 61.4 | 166.3 | 65.4 | 135.0 |
| [%] | | | | | |
| SEM AD | 20.8 | 5.9 | 18.7 | 12.5 | 12.8 |
| [%] | | | | | |
| p-value | 0.0776 | 0.0177 | 0.0207 | 0.0742 | 0.0520 |
| Cut-Off | <97 | <97 | >103 | <97 | >103 |
| [%] | | | | | |
| Accuracy | 67 | 73 | 73 | 67 | 67 |
| smallest | 69.83 | 14.22 | 14.11 | 16.77 | 7.04 |
| absolute | | | | | |
| change AD | | | | | |
| [%] | | | | | |

| Mass | | | | | | |
|---|---|---|---|---|---|---|
| [g/mol] | 331.2545 | 332.1372 | 334.2146 | 339.3135 | 346.1528 | 350.0956 |
| AD [%] | 74.0022047 | 47.7189049 | 18.7274459 | 238.408529 | 43.1569306 | 120.900326 |
| AD [%] | 58.447463 | 83.3772627 | 0.37324219 | 107.925549 | 93.1604667 | 100.833822 |
| AD [%] | 67.1660539 | 50.8143131 | 30.4145597 | 51.4270424 | 48.99405 | 102.250234 |
| AD [%] | 90.8354427 | 59.0410256 | 81.81283 | 259.307773 | 40.6626141 | 120.401339 |
| AD [%] | 33.2299836 | 102.74499 | 12.1961966 | 301.266842 | 71.3671719 | 154.771564 |
| AD [%] | 60.4537776 | 88.8034156 | 19.8769512 | 337.335838 | 66.0133488 | 125.896721 |
| AD [%] | 61.5609329 | 32.8045007 | 43.4198096 | 184.066249 | 29.0241151 | 161.487969 |
| AD [%] | 65.014587 | 35.4237217 | 28.3464043 | 171.308598 | 65.2927352 | 110.788654 |
| Healthy | 106.198807 | 87.220738 | 30.6793783 | 60.36895 | 58.9346409 | 100.852691 |
| [%] | | | | | | |
| Healthy | 80.7462596 | 56.5925018 | 70.5098865 | 353.846555 | 32.5145111 | 86.6677222 |
| [%] | | | | | | |
| Healthy | 89.5748208 | 76.7385744 | 306.540651 | 96.2932714 | 127.481956 | 132.753905 |
| [%] | | | | | | |
| Healthy | 105.059966 | 99.9532483 | 50.1156343 | 58.1465808 | 106.973533 | 88.2005524 |
| [%] | | | | | | |
| Healthy | 172.997054 | 167.169939 | 73.3919738 | 42.6629225 | 171.098221 | 106.345606 |
| [%] | | | | | | |
| Healthy | 98.9276396 | 92.4249075 | 112.822315 | 39.5942288 | 64.1384285 | 81.268202 |
| [%] | | | | | | |
| Healthy | 46.4954527 | 119.900091 | 55.9401608 | 49.0874914 | 138.858709 | 103.911321 |
| [%] | | | | | | |
| Mean Healthy | 100.0 | 100.0 | 100.0 | 100.0 | 100.0 | 100.0 |
| [%] | | | | | | |
| SEM Healthy | 14.4 | 13.4 | 35.7 | 42.9 | 18.8 | 6.5 |
| [%] | | | | | | |
| Mean AD | 63.8 | 62.6 | 29.4 | 206.4 | 57.2 | 124.7 |
| [%] | | | | | | |
| SEM AD | 5.7 | 9.2 | 8.8 | 34.2 | 7.3 | 8.0 |
| [%] | | | | | | |
| p-value | 0.0292 | 0.0351 | 0.0618 | 0.0716 | 0.0440 | 0.0354 |
| Cut-Off | <97 | <97 | <97 | >103 | <97 | >103 |
| [%] | | | | | | |
| Accuracy | 80 | 67 | 67 | 87 | 80 | 67 |
| smallest | 9.16 | 2.74 | 18.19 | 7.93 | 6.84 | 0.83 |
| absolute | | | | | | |
| change AD | | | | | | |
| [%] | | | | | | |

| Mass | | | | |
|---|---|---|---|---|
| [g/mol] | 355.345 | 358.159 | 366.0912 | 371.8758 |
| AD [%] | 170.842238 | 102.991005 | 118.00293 | 124.633366 |
| AD [%] | 156.915727 | 64.4515272 | 104.849559 | 95.3964806 |
| AD [%] | 161.754433 | 107.391245 | 103.277929 | 139.612426 |
| AD [%] | 119.400188 | 85.5519978 | 108.198659 | 110.413376 |
| AD [%] | 113.693434 | 68.6854054 | 133.858019 | 143.906204 |
| AD [%] | 138.379329 | 87.8996542 | 122.921113 | 115.512282 |
| AD [%] | 128.046581 | 88.0397563 | 96.0578605 | 94.4230477 |
| AD [%] | 95.317097 | 74.401814 | 125.094787 | 113.254251 |
| Healthy | 144.119951 | 107.749031 | 107.726449 | 110.073446 |
| [%] | | | | |
| Healthy | 121.122453 | 121.97295 | 100.246034 | 93.7050269 |
| [%] | | | | |
| Healthy | 84.2619566 | 102.058638 | 88.9862391 | 98.1246711 |
| [%] | | | | |
| Healthy | 71.8100794 | 92.0070678 | 97.1162985 | 109.385682 |
| [%] | | | | |
| Healthy | 87.2809675 | 83.7574603 | 105.902391 | 87.9823233 |
| [%] | | | | |
| Healthy | 112.576122 | 95.0897769 | 106.316009 | 91.8313161 |
| [%] | | | | |
| Healthy | 78.8284703 | 97.3650764 | 93.7065789 | 108.897535 |
| [%] | | | | |
| Mean Healthy | 100.0 | 100.0 | 100.0 | 100.0 |
| [%] | | | | |
| SEM Healthy | 10.0 | 4.6 | 2.7 | 3.5 |
| [%] | | | | |
| Mean AD | 135.5 | 84.9 | 114.0 | 117.1 |
| [%] | | | | |
| SEM AD | 9.3 | 5.4 | 4.6 | 6.4 |
| [%] | | | | |
| p-value | 0.0217 | 0.0579 | 0.0242 | 0.0435 |
| Cut-Off | >103 | <97 | >103 | >103 |
| [%] | | | | |
| Accuracy | 73 | 67 | 73 | 67 |
| smallest | 4.68 | 2.99 | 3.28 | 4.60 |
| absolute | | | | |
| change AD | | | | |
| [%] | | | | |

| Mass | | | | | |
|---|---|---|---|---|---|
| [g/mol] | 440.2751 | 538.1752 | 540.2563 | 702.5682 | 782.5638 |
| AD [%] | 42.5072218 | 131.080491 | 134.200918 | 110.385804 | 77.4187824 |
| AD [%] | 47.2458583 | 110.959175 | 123.667045 | 158.774306 | 97.3391226 |
| AD [%] | 56.8523297 | 127.436707 | 180.148162 | 135.602828 | 61.6100522 |
| AD [%] | 37.163682 | 130.918196 | 186.597897 | 128.159962 | 48.3292933 |
| AD [%] | 28.8325527 | 145.135632 | 172.054228 | 100.341527 | 93.8742131 |
| AD [%] | 97.8328606 | 137.586619 | 103.167342 | 105.658111 | 58.5030359 |
| AD [%] | 121.312184 | 88.3924107 | 133.405377 | 108.055361 | 69.9260889 |
| AD [%] | 55.6257328 | 150.800406 | 260.227236 | 124.91101 | 63.1706767 |
| Healthy | 99.3307029 | 105.377088 | 166.369749 | 115.733649 | 65.3296722 |
| [%] | | | | | |
| Healthy | 87.180806 | 85.2133473 | 123.780328 | 117.185117 | 89.0436545 |
| [%] | | | | | |
| Healthy | 125.869527 | 93.3614522 | 97.5434107 | 104.777033 | 101.779382 |
| [%] | | | | | |
| Healthy | 94.1090327 | 110.110467 | 64.2417557 | 85.0691926 | 130.009422 |
| [%] | | | | | |
| Healthy | 157.08456 | 116.276722 | 93.3919988 | 89.7497091 | 66.503633 |
| [%] | | | | | |
| Healthy | 94.5107159 | 102.454565 | 81.4680133 | 106.52984 | 106.372596 |
| [%] | | | | | |
| Healthy | 41.9146553 | 87.2063588 | 73.2047436 | 80.9554607 | 140.96164 |
| [%] | | | | | |
| Mean Healthy | 100.0 | 100.0 | 100.0 | 100.0 | 100.0 |
| [%] | | | | | |
| SEM Healthy | 13.4 | 4.4 | 13.2 | 5.6 | 11.0 |
| [%] | | | | | |
| Mean AD | 60.9 | 127.8 | 161.7 | 121.5 | 71.3 |
| [%] | | | | | |
| SEM AD | 11.3 | 7.0 | 17.5 | 6.9 | 6.1 |
| [%] | | | | | |
| p-value | 0.0428 | 0.0067 | 0.0168 | 0.0331 | 0.0341 |
| Cut-Off | <94 | >103 | >103 | >103 | <97 |
| [%] | | | | | |
| Accuracy | 73 | 73 | 87 | 67 | 73 |
| smallest | 2.17 | 10.96 | 3.17 | 0.34 | 2.66 |
| absolute | | | | | |
| change AD | | | | | |
| [%] | | | | | |

### 4. Screening for Mass Combinations Predictive of Disease Status

To increase accuracy and robustness of this diagnostic approach, the final dataset was further analyzed and processed utilizing supervised machine-learning algorithms to evaluate the diagnostic potential of all possible combinations of up to 7 masses (excluding combinations that would include highly correlating masses) and extract combinations of high diagnostic value. Because of the small cohort size a rigorous approach of training and testing classifier models have been utilized to estimate the diagnostic robustness of a specific mass combination against unknown samples.

A scikit-learn GridSearch pipeline with a nested cross-validation approach was utilized to evaluate the best mass combinations and classifiers for identifying AD patients. Within the GridSearch pipeline, the following supervised learning methods have been screened: Logistic Regression, Stochastic Gradient Descent Classifier (SGDClassifier), Decision Tree Classifier, Random Tree Classifier, Extra Tree Classifier, AdaBoost Classifier, Multi-layer Perceptron Classifier (MLPClassifier), Gradient Boosting Classifier, Gaussian Naïve Bayes (GaussianNB), K-Nearest Neighbor Classifier, C-Support Vector Classification (SVC), Quadratic Discriminant Analysis and Linear Regression. For each method, hyperparameter optimization and training were performed on 70% of the full data set using a stratified three-fold data split (inner loop). The classifier with the highest area under the ROC curve (AUC) was then evaluated on the remaining 30% of the data set using the leave-one-out method (outer loop) to assess the generalizability and accuracy of the model and mass combination.

### Results

The screening revealed a large number of combinations of 1 to 7 of the 45 found masses that were most valuable for accurate prediction of the disease status. Table 2 below shows an exemplary selection of mass combinations that predicted the disease status with high accuracy. Because the calculation of predictions of most classifiers was hard or not at all to depict, only logistic regression algorithms were chosen for this table 2. However, in most cases, the decisive factor for accurate prediction was the combination of masses and not the algorithm used for the prediction, which again highlights the validity of the selected masses. This fact was exemplified in table 4, where key diagnostic parameters are shown for different classifiers using the same mass combinations.

For disease prognosis, logistic regression equations are to be interpreted as follows:

y=I+C₁*F₁+C₂*F₂+ . . . +Cₙ*Fₙ,
where I is the Intercept and Cₙ the coefficient of Mass Fₙ. Positive y-values are a prediction of Alzheimer's disease while negative values predict the non-diseased status. Greater absolute values mean great certainty of the prediction.

For example, in case of the first combination of masses shown in table 2 the formula was as follows:

y=19,61932517+(−64,96994161*[Feat1])+(43,45875272*[Feat2])+(−60,67815063*[Feat3])+(47,81007498*[Feat4])
where [FeatX] was the concentration (standard scaled or absolute value) of the respective mass in a sample.

TABLE 2

| | | | | | | | | Cut-offs for | | |
|---|---|---|---|---|---|---|---|---|---|---|
| Examplary combination(s) | | | | | | | | AD Diagnosis | | |
| Feat1 | Feat2 | Feat3 | Feat4 | Feat5 | Feat6 | Feat7 | MCA | Var A | Var B | Var C/D |
| 110.0367 | 254.1153 | 189.0096 | 224.084 | | | | 1.00 | >99% | >103% | >0 |
| 232.004 | 254.1153 | 189.9936 | 224.084 | | | | 1.00 | >99% | >103% | >0 |
| 232.004 | 224.084 | 702.5682 | 315.2409 | 175.0633 | | | 1.00 | >99% | >103% | >0 |
| 254.1153 | 175.0633 | 208.0737 | | | | | 1.00 | >87.5% | >103% | >0 |
| 110.0367 | 254.1153 | 224.084 | 315.2409 | 175.0633 | | | 1.00 | >99% | >103% | >0 |
| 702.5682 | 315.2409 | 175.0633 | 208.0737 | | | | 1.00 | >87.5% | >112% | >0 |
| 110.0367 | 315.2409 | 175.0633 | | | | | 1.00 | >98% | >103% | >0 |
| 110.0367 | 232.004 | 315.2409 | 208.0737 | | | | 1.00 | >99% | >103% | >0 |
| 175.0633 | 208.0737 | | | | | | 1.00 | >87.5% | >103% | >0 |
| 110.0367 | 175.0633 | 208.0737 | | | | | 1.00 | >99% | >103% | >0 |
| 702.5682 | 208.0737 | | | | | | 1.00 | >99% | >112% | >0 |
| 175.0633 | 208.0737 | 315.2409 | | | | | 1.00 | <60% | >103% | >0 |
| 110.0367 | 232.004 | 254.1153 | 189.9936 | 189.0096 | 224.084 | | 1.00 | >99% | >103% | >0 |
| 110.0367 | 175.0633 | 702.5682 | 208.0737 | 315.2409 | 232.004 | 254.1153 | 1.00 | >98% | >103% | >0 |

| | Intercept + Coefficents (belonging to each | | | | | | | |
|---|---|---|---|---|---|---|---|---|
| Formula | feature) to be used in the logistic formula | | | | | | | |
| Accuracy | Intercept | Coeff1 | Coeff2 | Coeff3 | Coeff4 | Coeff5 | Coeff6 | Coeff7 |
| 1.00 | 19.61933 | −64.96994 | 43.45875 | −60.67815 | 47.81007 | | | |
| Ranges | 50 | 150 | 100 | 150 | 100 | | | |
| | to −50 | to −150 | to −100 | to −150 | to −100 | | | |
| 1.00 | −0.027339 | −59.6902 | 18.03567 | −4.467575 | 47.94471 | | | |
| Ranges | 50 | 150 | 100 | 100 | 100 | | | |
| | to −50 | to −150 | to −100 | to −100 | to −100 | | | |
| 1.00 | −35.38932 | −240.2359 | 77.96284 | 100.8236 | 83.30664 | 45.55984 | | |
| Ranges | 50 | 300 | 150 | 150 | 150 | 100 | | |
| | to −50 | to −300 | to −150 | to −150 | to −150 | to −100 | | |
| 1.00 | −0.193003 | 0.630932 | 1.051046 | −0.86848 | | | | |
| Ranges | 50 | 100 | 100 | 100 | | | | |
| | to −50 | to −100 | to −100 | to −100 | | | | |
| 1.00 | 17.85179 | −43.26595 | 42.88328 | 15.24719 | 4.194449 | 41.28051 | | |
| Ranges | 50 | 150 | 150 | 150 | 100 | 150 | | |
| | to −50 | to −150 | to −150 | to −150 | to −100 | to −150 | | |
| 1.00 | −17.76068 | 15.77153 | 27.06462 | 13.59515 | −53.66368 | | | |
| Ranges | 50 | 100 | 100 | 100 | 150 | | | |
| | to −50 | to −100 | to −100 | to −100 | to −150 | | | |
| 0.93 | −0.13616 | −33.05911 | −24.65771 | 46.70401 | | | | |
| Ranges | 50 | 250 | 100 | 220 | | | | |
| | to −50 | to −250 | to −100 | to −220 | | | | |
| | 0.028405 | −17.35242 | −42.90811 | 9.113668 | −3.697252 | | | |
| Ranges | 50 | 150 | 300 | 150 | 100 | | | |
| | to −50 | to −150 | to −300 | to −150 | to −100 | | | |
| 0.80 | 3.7E−05 | −0.019711 | −0.027794 | | | | | |
| Ranges | 50 | 50 | 60 | | | | | |
| | to −50 | to −50 | to −60 | | | | | |
| 0.87 | 0.416279 | 0.155179 | −0.596613 | −0.962069 | | | | |
| Ranges | 50 | 70 | 60 | 60 | | | | |
| | to −50 | to −70 | to −60 | to −60 | | | | |
| 0.73 | −2.056652 | 7.8824 | −3.032653 | | | | | |
| Ranges | 50 | 100 | 60 | | | | | |
| | to −50 | to −100 | to −60 | | | | | |
| 0.80 | 0.000232 | −0.036766 | −0.035076 | −0.000276 | | | | |
| Ranges | 50 | 100 | 100 | 400 | | | | |
| | to −50 | to −100 | to −100 | to −400 | | | | |
| 1.00 | −3.0929 | −174.4709 | −142.8151 | 19.4019 | −8.8774 | −97.7147 | 105.7223 | |
| Ranges | 50 | 220 | 250 | 100 | 200 | 250 | 200 | |
| | to −50 | to −220 | to −250 | to −100 | to −200 | to −250 | to −200 | |
| 0.93 | 17.7954 | 50.3966 | −84.7399 | 11.9336 | −50.8482 | 54.5133 | −26.0136 | −7.006 |
| Ranges | 50 | 100 | 150 | 150 | 100 | 120 | 60 | 60 |
| | to −50 | to −100 | to −150 | to −150 | to −100 | to −120 | to −60 | to −60 |

Feat: Feature
Coeff: Coefficient
MAC: Maximum Accuracy found for this Combination of masses
All value ranges indicated in the table for “Ranges” are meant as excluding zero.

Without being bound to this theory, it is assumed that of the 45 compounds indicated by a mass in gram/mol above, the following 5 compounds are associated with chemical compounds as listed below in Table 3:

TABLE 3
Compounds having specific mass in
gram/mol associated with structures

| Mass [g/mol] | Chemical name |
|---|---|
| 110.0367 | Catechol |
| 208.0737 | 3,4-dimethoxycinnamic acid |
| 315.2409 | Decanoylcarnitine |
| 175.0633 | Indole-3-acetic acid |
| 702.5682 | Palmitoyl sphingomyelin |

TABLE 4

| | | | | | | | Sensi- | Speci- | | |
|---|---|---|---|---|---|---|---|---|---|---|
| Feat1 | Feat2 | Feat3 | Feat4 | Feat5 | Feat6 | Accuracy | titivty | fitcity | Precision | Classifier |
| 110.0367 | 232.004 | 254.1153 | 189.9936 | 89.0096 | 224.084 | 1.00 | 1.00 | 1.00 | 1.00 | AdaBoostClassifier |
| | | | | | | 1.00 | 1.00 | 1.00 | 1.00 | DecisionTreeClassifier |
| | | | | | | 1.00 | 1.00 | 1.00 | 1.00 | ExtraTreesClassifier |
| | | | | | | 0.93 | 0.86 | 1.00 | 1.00 | GaussianNB |
| | | | | | | 1.00 | 1.00 | 1.00 | 1.00 | GaussianPro- |
| | | | | | | | | | | cessClassifier |
| | | | | | | 1.00 | 1.00 | 1.00 | 1.00 | GradientBoostingClas- |
| | | | | | | | | | | sifier |
| | | | | | | 1.00 | 1.00 | 1.00 | 1.00 | KNeighborsClassifier |
| | | | | | | 1.00 | 1.00 | 1.00 | 1.00 | LinearRegression |
| | | | | | | 0.93 | 0.86 | 1.00 | 1.00 | LogisticRegression |
| | | | | | | 1.00 | 1.00 | 1.00 | 1.00 | MLPClassifier |
| | | | | | | 1.00 | 1.00 | 1.00 | 1.00 | QuadraticDiscrim- |
| | | | | | | | | | | inantAnalysis |
| | | | | | | 1.00 | 1.00 | 1.00 | 1.00 | RandomForestClassifier |
| | | | | | | 1.00 | 1.00 | 1.00 | 1.00 | SGDClassifier |
| | | | | | | 0.93 | 0.86 | 1.00 | 1.00 | SVC |
| 110.0367 | 315.2409 | 175.0633 | | | | 1.00 | 1.00 | 1.00 | 1.00 | AdaBoostClassifier |
| | | | | | | 1.00 | 1.00 | 1.00 | 1.00 | DecisionTreeClassifier |
| | | | | | | 1.00 | 1.00 | 1.00 | 1.00 | ExtraTreesClassifier |
| | | | | | | 0.80 | 0.86 | 0.75 | 0.75 | GaussianNB |
| | | | | | | 0.87 | 0.86 | 0.88 | 0.86 | GaussianPro- |
| | | | | | | | | | | cessClassifier |
| | | | | | | 1.00 | 1.00 | 1.00 | 1.00 | GradientBoostingClas- |
| | | | | | | | | | | sifier |
| | | | | | | 1.00 | 1.00 | 1.00 | 1.00 | KNeighborsClassifier |
| | | | | | | 0.73 | 0.71 | 0.75 | 0.71 | LinearRegression |
| | | | | | | 0.87 | 0.86 | 0.88 | 0.86 | LogisticRegression |
| | | | | | | 0.87 | 0.86 | 0.88 | 0.86 | MLPClassifier |
| | | | | | | 0.80 | 0.71 | 0.88 | 0.83 | QuadraticDiscrim- |
| | | | | | | | | | | inantAnalysis |
| | | | | | | 1.00 | 1.00 | 1.00 | 1.00 | RandomForestClassifier |
| | | | | | | 0.93 | 0.86 | 1.00 | 1.00 | SGDClassifier |
| | | | | | | 0.87 | 0.86 | 0.88 | 0.86 | SVC |
| 702.5682 | 208.0737 | 315.2409 | | | | 0.87 | 0.71 | 1.00 | 1.00 | AdaBoostClassifier |
| | | | | | | 1.00 | 1.00 | 1.00 | 1.00 | DecisionTreeClassifier |
| | | | | | | 1.00 | 1.00 | 1.00 | 1.00 | ExtraTreesClassifier |
| | | | | | | 0.87 | 1.00 | 0.75 | 0.78 | GaussianNB |
| | | | | | | 0.93 | 0.86 | 1.00 | 1.00 | GaussianPro- |
| | | | | | | | | | | cessClassifier |
| | | | | | | 1.00 | 1.00 | 1.00 | 1.00 | GradientBoostingClas- |
| | | | | | | | | | | sifier |
| | | | | | | 1.00 | 1.00 | 1.00 | 1.00 | KNeighborsClassifier |
| | | | | | | 0.73 | 0.71 | 0.75 | 0.71 | LinearRegression |
| | | | | | | 0.73 | 0.71 | 0.75 | 0.71 | LogisticRegression |
| | | | | | | 0.80 | 0.71 | 0.88 | 0.83 | LogisticRegression |
| | | | | | | 0.73 | 0.71 | 0.75 | 0.71 | MLPClassifier |
| | | | | | | 0.87 | 1.00 | 0.75 | 0.78 | QuadraticDiscrim- |
| | | | | | | | | | | inantAnalysis |
| | | | | | | 1.00 | 1.00 | 1.00 | 1.00 | RandomForestClassifier |
| | | | | | | 0.73 | 0.57 | 0.88 | 0.80 | SVC |
| 189.9936 | 110.0367 | 224.084 | 189.0096 | | | 1.00 | 1.00 | 1.00 | 1.00 | AdaBoostClassifier |
| | | | | | | 1.00 | 1.00 | 1.00 | 1.00 | DecisionTreeClassifier |
| | | | | | | 1.00 | 1.00 | 1.00 | 1.00 | ExtraTreesClassifier |
| | | | | | | 0.87 | 0.86 | 0.88 | 0.86 | GaussianNB |
| | | | | | | 0.93 | 0.86 | 1.00 | 1.00 | GaussianPro- |
| | | | | | | | | | | cessClassifier |
| | | | | | | 1.00 | 1.00 | 1.00 | 1.00 | GradientBoostingClas- |
| | | | | | | | | | | sifier |
| | | | | | | 1.00 | 1.00 | 1.00 | 1.00 | KNeighborsClassifier |
| | | | | | | 0.87 | 0.86 | 0.88 | 0.86 | LinearRegression |
| | | | | | | 0.80 | 0.86 | 0.75 | 0.75 | LogisticRegression |
| | | | | | | 1.00 | 1.00 | 1.00 | 1.00 | MLPClassifier |
| | | | | | | 0.93 | 0.86 | 1.00 | 1.00 | QuadraticDiscrim- |
| | | | | | | | | | | inantAnalysis |
| | | | | | | 1.00 | 1.00 | 1.00 | 1.00 | RandomForestClassifier |
| | | | | | | 1.00 | 1.00 | 1.00 | 1.00 | SGDClassifier |
| | | | | | | 0.93 | 0.86 | 1.00 | 1.00 | \|SVC |

### REFERENCES

[1] US 2015/0374711 A1.
[2] Schindler S E, Bollinger J G, Ovod V, et al. High-precision plasma beta-amyloid 42/40 predicts current and future brain amyloidosis. Neurology 2019, 93 (17): e1647-e1659.
[3] Eugenia Trushina E, Dutta T, Persson X-M T, Mielke M M, Petersen R C, Identification of Altered Metabolic Pathways in Plasma and CSF in Mild Cognitive Impairment and Alzheimer's Disease Using Metabolomics. PlosOne, May 2013, Volume 8, Issue 5, e63644.
[4] Dunn W B, Broadhurst D, Begley P, et al. Procedures for large-scale metabolic profiling of serum and plasma using gas chromatography and liquid chromatography coupled to mass spectrometry. Nature Protocols, 2011, 6 (7): 1060-1083.
[5] Sarafian M H, Gaudin M, Lewis M R, et al. Objective Set of Criteria for Optimization of Sample Preparation Procedures for Ultra-High Throughput Untargeted Body fluid Plasma Lipid Profiling by Ultra Performance Liquid Chromatography-Mass Spectrometry. Analytical Chemistry, 2014, 86 (12): 5766-5774.
[6] Guida R D, Engel J, Allwood J W, et al. Non-targeted UHPLC-MS metabolomic data processing methods: a comparative investigation of normalisation, missing value imputation, transformation and scaling [J]. Metabolomics, 2016, 12 (5): 93.
[7] Barker, M.; Rayens, W., Partial least squares for discrimination. J Chemometr 2003, 17 (3): 166-173.
[8] Westerhuis J A, Hoefsloot H C J, Smit S, et al. Assessment of PLSDA cross validation [J]. Metabolomics, 2008, 4 (1): 81-89.

1. A method for identifying a subject suffering from Alzheimer's dementia (AD) or being at risk of developing Alzheimer's dementia, the method comprising:
(a) determining in a first body fluid sample of the subject the amount of at least a compound having a mass in gram/mol of 110.0367;
(b) determining in a second body fluid sample of the subject the amount of the same compound as in (a);
(c) determining the change of the amount of the compound determined according to (a) compared to the respective amount of the compound determined according to (b);
(d) analyzing the change as determined in (c) using a computer-implemented prediction algorithm that is capable of predicting AD in a subject based on the compound, whereby a subject suffering from AD or being at risk of developing Alzheimer's dementia is identified if AD is predicted;
wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

2. The method for identifying a subject suffering from Alzheimer's dementia (AD) or being at risk of developing Alzheimer's dementia according to claim 1, wherein the first body fluid sample derives from a subject after the subject had been administered thiethylperazine or a pharmaceutically acceptable salt thereof.

3. The method for identifying a subject suffering from Alzheimer's dementia (AD) or being at risk of developing Alzheimer's dementia according to claim 1or 2, wherein the amount of at least one further compound having a mass in gram/mol selected from the group consisting of 208.0737, 315.2409, 175.0633, 702.5682 (Group 4), is additionally determined in a first body fluid sample of the subject in steps (a) and additionally analysed in (b).

4. The method for identifying a subject suffering from Alzheimer's dementia (AD) or being at risk of developing Alzheimer's dementia according to any one of claims 1 to 3, wherein said computer-implemented prediction algorithm uses a supervised machine-learning algorithm that has been trained by a training dataset comprising quantitative or qualitative data for
the compound having a mass in gram/mol of 110.0367, optionally in combination with the at least one further compound from Group 4,
in body fluid samples from subjects known to suffer from AD and subjects known not to suffer from AD.

5. The method according to any one of claims 1 to 4, wherein at least the amounts of a compound having a mass of 110.0367 gram/mol, a compound having a mass of 175.0633 gram/mol, and a compound having a mass of 208.0737 gram/mol are determined in (a) and analyzed in (b).

6. A method for identifying a subject suffering from Alzheimer's dementia (AD) or being at risk of developing Alzheimer's dementia, the method comprising:
(a) determining in a first body fluid sample of the subject the amount of a compound having a mass in gram/mol of 110.0367;
(b) determining in a second body fluid sample of the subject the amount of the same compound as in (a);
(c) comparing the amount of the compound determined according to (a) to the respective amount of the compound determined according to (b), wherein a change of the amount of the compound according to (b) compared with the amount as determined in (a) indicates Alzheimer's dementia for the subject or the risk of developing Alzheimer's dementia for the subject;
wherein said first body fluid sample had been obtained prior to administration of thiethylperazine or a pharmaceutically acceptable salt thereof and said second body fluid sample had been obtained after the subject had been administered thiethylperazine or a pharmaceutically acceptable salt thereof.

7. A method for identifying a subject suffering from Alzheimer's dementia (AD), the method comprising:
(a) determining in a body fluid sample of the subject the amount of a compound having a mass in gram/mol of 110.0367
(b) comparing the amount of the compound determined according to (a) to a mean amount of a healthy subject, wherein a change of the amount of the compound according to (a) compared with the mean amount of a healthy subject, preferably by at least 3% based on the mean amount of a healthy subject being 100%, indicates Alzheimer's dementia for the subject or indicates that the subject is at risk of developing Alzheimer's dementia;
wherein said body fluid sample had been obtained after the subject had been administered thiethylperazine or a pharmaceutically acceptable salt thereof.

8. Compound having a mass in gram/mol of 110.0367 and optionally in combination with at least one compound having a mass in gram/mol selected from Group 4, for diagnosing AD in a subject or for determining the risk of a subject to develop AD.

9. Use of at least one compound having a mass in gram/mol of 110.0367 and optionally in combination with at least one compound having a mass in gram/mol selected from Group 4 in a sample of a subject for diagnosing AD in said subject or for determining the risk of said subject to develop AD.

10. A device adapted for identifying a subject suffering from Alzheimer's dementia (AD) or being at risk of developing Alzheimer's dementia, the device comprising:
(a) an analyzing unit capable of determining in a first body fluid sample of the subject the amount of at least one compound having a mass in gram/mol of 110.0367;
(b) a data processor adopted for analyzing the determined amount for the compound using a prediction algorithm implemented in said data processor that is capable of predicting AD in a subject based on the at least two compounds, whereby a subject suffering from AD is identified if AD is predicted or a subject being at risk of developing Alzheimer's dementia is identified if AD is predicted.

11. A kit for identifying a subject suffering from Alzheimer's dementia (AD) or being at risk of developing Alzheimer's dementia, the kit comprising a reference sample of a compound having a mass in gram/mol of 110.0367, optionally a reference sample of at least one compound having a mass in gram/mol selected from the group consisting of Group 4, and optionally one or more reagents for sample preparation and/or extraction.
